(19) United States
(12) Patent Application Publication
(10) Pub. No.: US 2023/0046062 A1
(43) Pub. Date: Feb. 16, 2023

(54) ANTIMICROBIAL COMPOSITIONS AND METHODS OF USING THEREOF

(71) Applicants: Ohio State Innovation Foundation, Columbus, OH (US); ZEOVATION, INC., Columbus, OH (US)

(72) Inventors: Prabir K. DUTTA, Columbus, OH (US); Bo WANG, Ashburn, VA (US); James WALDMAN, Columbus, OH (US); Deborah KNIGHT, Columbus, OH (US); Sweta SHRESTHA, Hilliard, OH (US)

(21) Appl. No.: 17/782,088

(22) PCT Filed: Dec. 2, 2020
(86) PCT No.: PCT/US2020/062913

§ 371 Date: Jun. 2, 2022

Related U.S. Application Data
(60) Provisional application No. 63/015,255, filed on Apr. 24, 2020.
(60) Provisional application No. 62/943,172, filed on Dec. 3, 2019.
(60) Provisional application No. 62/942,740, filed on Dec. 2, 2019.

(51) Int. Cl. A01N 59/20; A01N 25/04; A01N 25/34; A61L 15/18; A61L 15/44; D06M 16/00

(52) U.S. Cl. CPC A01N 59/20; A01N 25/04; A01N 25/34; A61L 15/18; A61L 15/44; D06M 16/00; B82Y 30/00

(57) ABSTRACT

Disclosed herein are compositions (e.g., sprays, paints, etc.) that comprise antimicrobial zeolite nanoparticles. Also provided are hemostatic compositions comprising zeolite nanoparticles, dryer sheets comprising zeolite nanoparticles, and textiles comprising zeolite nanoparticles. Also disclosed are compositions (e.g., sprays) that include a binder polymer to improve coating adherence. In some cases, the zeolite nanoparticles can further comprise an optical tracer (e.g., a fluorophore) associated with the zeolite nanoparticles. The optical tracer can be interrogated to confirm presence of the zeolite nanoparticles (or a coating comprising the zeolite nanoparticles) on a surface. Also provided are methods of forming viricidal coatings using compositions that comprise zeolite nanoparticles dispersed in a carrier.

### CROSS-REFERENCE TO RELATED APPLICATIONS

This application claims benefit of U.S. Provisional Application No. 62/942,740, filed Dec. 2, 2019, U.S. Provisional Application No. 62/943,172, filed Dec. 3, 2019, and U.S. Provisional Application No. 63/015,255, filed Apr. 24, 2020, each of which is hereby incorporated by reference in its entirety.

### BACKGROUND

A number of inorganic materials have been shown to possess antimicrobial activity. They include metal ions such as silver, copper, zinc, mercury, tin, lead, bismuth, cadmium, chromium and thallium ions. It is theorized that these antimicrobial metal ions exert their effects by disrupting respiration and electron transport systems upon absorption into bacterial or fungal cells. Antimicrobial metal ions of silver, copper, zinc, and gold, in particular, are considered safe for in vivo use. Antimicrobial silver ions are particularly useful due to the fact that they exhibit a high ratio of efficacy to toxicity.

Antimicrobial zeolites have been prepared by replacing all or part of the ion-exchangeable ions in zeolite with antimicrobial metal ions, as described in U.S. Pat. Nos. 4,911,898; 4,911,899; 4,938,955; 4,906,464; and 4,775,585. However, existing antimicrobial zeolites suffer from shortcomings that have thus far limited their widespread adoption. Accordingly, improved antimicrobial zeolites, as well as improved compositions and methods for using such zeolites, are needed.

### SUMMARY

Provided herein are antimicrobial composition comprising water and a population of zeolite nanoparticles dispersed therein. The zeolite nanoparticles can comprise an effective amount of antimicrobial metal ions to kill or inhibit the growth of a microbe. In some 30 embodiments, the composition can further comprise an optical tracer associated with the zeolite nanoparticles. In some embodiments, the composition can further comprise a binder polymer. The composition can be applied to a surface to form an antimicrobial coating on the surface. If desired, the optical tracer (when present) can be interrogated to determine whether the coating comprising the zeolite nanoparticles remains resident on the surface. In this way, the stability of the antimicrobial coating can be readily assessed, allowing for worn or degraded coatings to be reapplied (so as to main adequate antimicrobial protection on surfaces). The binder polymer (when present) can improve the abrasion resistance and stability of the coating.

The zeolite nanoparticles can comprise a surface that has been modified via association of a hydrophobic capping molecule. In some embodiments, the capping molecule can comprise a hydrophobic molecule comprising a cationic moiety, and wherein the cationic moiety is electrostatically associated with the surface of the zeolite.

The capping molecule can comprise an amine defined by Formula I or Formula II below

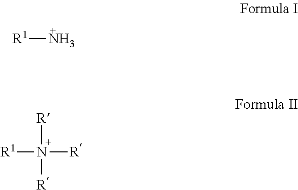

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the surface of the nanozeolites can be covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

In some embodiments, the surface of the nanozeolites can be covalently modified via reaction with a halosilane selected from octadecyltrichlorosilane (OTS), hexyltrichlorosilane (HTS), ethyltrichlorosilane (ETS), and combinations thereof.

In some embodiments, the zeolite nanoparticles can have an average diameter of less than 100 nm, such as from 10 nm to less than 100 nm, or from 20 nm to 60 nm.

In some embodiments, the antimicrobial metal ions comprise metal nanoparticles formed from an antimicrobial metal. The antimicrobial metal can comprise, for example, silver, copper, zinc, or a combination thereof. In some cases, the metal nanoparticles can have an average diameter of 10 nm or less, such as from 1 nm to 10 nm, or from 1 nm to 5 nm. In some cases, the metal nanoparticles can be present in an amount of at least 1% by weight, based on the total weight of the zeolite nanoparticles and the metal nanoparticles, such as from 1% to 25% by weight, based on the total weight of the zeolite nanoparticles and the metal nanoparticles.

In some embodiments, the antimicrobial metal ions can comprise antimicrobial metal ions retained at ion-exchangeable sites within the zeolite nanoparticles. The antimicrobial metal can comprise, for example, silver, copper, zinc, or a combination thereof. The antimicrobial metal ions can be present in an amount of 10% or greater of the ion exchange capacity of the zeolite nanoparticles, such as from 50% up to 100% of the ion exchange capacity of the zeolite nanoparticles.

In some embodiments, the zeolite nanoparticles can have an average internal surface area of at least 300 m²/g.

In some embodiments, the zeolite nanoparticles can further comprise an adjuvant, such as a small molecule antimicrobial agent.

The zeolite nanoparticles can be present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

In some embodiments, the composition can further comprise a non-ionic or zwitterionic surfactant. The non-ionic or zwitterionic surfactant can be present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

In some embodiments, the composition can further comprise a binder polymer dissolved or dispersed in the water. The binder polymer can help improve stability and durability of the zeolite nanoparticle coating on a surface. The binder polymer can comprise a water-soluble polymer. For example, the binder polymer can comprise a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof; hemicellulose or derivatives thereof, alginate; tetramethylene ether glycol; polyvinyl pyrrolidone; polyvinyl esters such as polyvinyl acetate; copolymers thereof; and mixtures thereof. The binder polymer can be present in an amount of 10% by weight or less, such as from 0.1% by weight to 10% by weight or from 0.1% to 5% by weight, based on the total weight of the composition.

In some embodiments, the composition can further comprise an optical tracer associated with the zeolite nanoparticles. In some cases, the optical tracer can be covalently bound to the zeolite nanoparticles. In some cases, the optical tracer can be non-covalently associated with the zeolite nanoparticles. In some cases, the optical tracer can comprise a fluorophore, such as a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.

Also provided are methods of producing a coating on a surface that comprise applying a composition described herein to the surface; and allowing the composition to dry to produce the coating.

The coating can kill or inhibit the growth of a microbe, such as a bacteria, a fungi, a virus, an algae, or a combination thereof. In some embodiments, the microbe is a bacteria, such as Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas fourescens, Nocardia brasiliensis, Nocardia globerula, Acinetobacter genomospecies, Acinetobacter calcoaceticus, Acinetobacter baumannii, Stenotrophomonas maltophlia, Pantoea stewarti ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof. In some embodiments, the microbe is a fungi, such as Candida albicans, Candida parapsilosis, Candida tropicalis, Candida glabrata, Kluyveromyces marxianus, Hyphopichia burtani, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof. In some embodiments, the microbe is a coronavirus, such as SARS-CoV-2.

In some embodiments, the composition comprises a binder polymer. In these embodiments, the coating can exhibit improved resistance to abrasion as compared to a coating formed from an otherwise identical composition in which the binder polymer is absent.

In some embodiments, the composition comprises an optical tracer associated with the zeolite nanoparticles. In these embodiments, the method can further comprise interrogating the optical tracer to determine when the coating has been worn from the surface. Interrogating the optical tracer can comprise, for example, irradiating the surface with a UV light and visually observing fluorescence from the optical tracer to determine when the coating has been worn from the surface. Alternatively, an instrument (e.g., a photometer) can be used to measure the fluorescence from the optical tracer to determine when the coating has been worn from the surface. In some embodiments, methods can further comprise replying the coating upon a determination the coating has been worn from the surface.

Also provided are methods of forming a viricidal coating on a surface. These methods can comprise applying to the surface the composition comprising a population of zeolite nanoparticles dispersed in a carrier, wherein the zeolite nanoparticles comprise an effective amount of antimicrobial metal ions to kill or inactivate the virus; and allowing the composition to dry to produce the viricidal coating.

In some embodiments, the carrier can comprise water.

The zeolite nanoparticles can comprise a surface that has been modified via association of a hydrophobic capping molecule. In some embodiments, the capping molecule can comprise a hydrophobic molecule comprising a cationic moiety, and wherein the cationic moiety is electrostatically associated with the surface of the zeolite.

The capping molecule can comprise an amine defined by Formula I or Formula II below

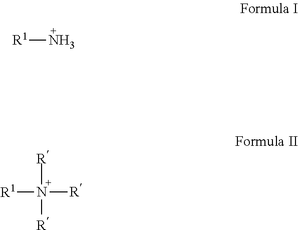

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the surface of the nanozeolites can be covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

In some embodiments, the surface of the nanozeolites can be covalently modified via reaction with a halosilane selected from octadecyltrichlorosilane (OTS), hexyltrichlorosilane (HTS), ethyltrichlorosilane (ETS), and combinations thereof.

In some embodiments, the zeolite nanoparticles can have an average diameter of less than 100 nm, such as from 10 nm to less than 100 nm, or from 20 nm to 60 nm.

In some embodiments, the antimicrobial metal ions comprise metal nanoparticles formed from an antimicrobial metal. The antimicrobial metal can comprise, for example, silver, copper, zinc, iron, or a combination thereof. In some cases, the metal nanoparticles can have an average diameter of 10 nm or less, such as from 1 nm to 10 nm, or from 1 nm to 5 nm. In some cases, the metal nanoparticles can be present in an amount of at least 1% by weight, based on the total weight of the zeolite nanoparticles and the metal nanoparticles, such as from 1% to 25% by weight, based on the total weight of the zeolite nanoparticles and the metal nanoparticles.

In some embodiments, the antimicrobial metal ions can comprise antimicrobial metal ions retained at ion-exchangeable sites within the zeolite nanoparticles. The antimicrobial metal can comprise, for example, silver, copper, zinc, iron, or a combination thereof. The antimicrobial metal ions can be present in an amount of 10% or greater of the ion exchange capacity of the zeolite nanoparticles, such as from 50% up to 100% of the ion exchange capacity of the zeolite nanoparticles.

In some embodiments, the zeolite nanoparticles can have an average internal surface area of at least 300 m²/g.

In some embodiments, the zeolite nanoparticles can further comprise an adjuvant, such as a small molecule antimicrobial agent.

The zeolite nanoparticles can be present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

In some embodiments, the composition can further comprise a non-ionic or zwitterionic surfactant. The non-ionic or zwitterionic surfactant can be present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

In some embodiments, the composition can further comprise a binder polymer dissolved or dispersed in the water. The binder polymer can help improve stability and durability of the zeolite nanoparticle coating on a surface. The binder polymer can comprise a water-soluble polymer. For example, the binder polymer can comprise a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof; hemicellulose or derivatives thereof, alginate; tetramethylene ether glycol; polyvinyl pyrrolidone; polyvinyl esters such as polyvinyl acetate; copolymers thereof; and mixtures thereof. The binder polymer can be present in an amount of 10% by weight or less, such as from 0.1% by weight to 10% by weight or from 0.1% to 5% by weight, based on the total weight of the composition.

In some embodiments, the composition can further comprise an optical tracer associated with the zeolite nanoparticles. In some cases, the optical tracer can be covalently bound to the zeolite nanoparticles. In some cases, the optical tracer can be non-covalently associated with the zeolite nanoparticles. In some cases, the optical tracer can comprise a fluorophore, such as a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.

In some embodiments, these methods can further comprise contacting the surface with an oxidant, such as hydrogen peroxide or chlorine oxide. This can comprise applying hydrogen peroxide as an aerosol to the surface (e.g., by spraying hydrogen peroxide vapor into airspace in contact with the coating). Transition metal ions (e.g., iron ions, copper ions, or a combination thereof) present in the zeolite nanoparticle can induce the formation of reactive radicals which kill or inhibit microbes that contact the surface.

Also provided are antimicrobial compositions that comprise water and a population of modified zeolite nanoparticles dispersed therein. The zeolite nanoparticles comprise an effective amount of antimicrobial metal ions to kill or inhibit the growth of a microbe.

The zeolite nanoparticles can be hydrophobically modified. The modified zeolite nanoparticles can comprise a surface that has been modified via association of a hydrophobic capping molecule. For example, the hydrophobic capping molecule can comprise a hydrophobic molecule comprising a cationic moiety, and wherein the cationic moiety is electrostatically associated with the surface of the zeolite. For example, the capping molecule can comprise an amine defined by Formula I or Formula II below

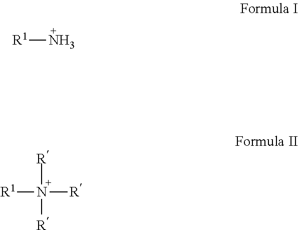

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the modified zeolite nanoparticles can comprise a surface that is covalently modified via reaction with an alkoxysilane, for example an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

In some embodiments, the modified zeolite nanoparticles can comprise a surface that is covalently modified via reaction with an halosilane, for example a halosilane selected from octadecyltrichlorosilane (OTS), hexyltrichlorosilane (HTS), ethyltrichlorosilane (ETS), and combinations thereof.

The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possesses a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The zeolite nanoparticles can be present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

In some embodiments, the composition can further comprise a surfactant (e.g., a non-ionic or zwitterionic surfactant, such as Triton X, Igepal, Tween, Brij). The surfactant can be present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

In some embodiments, the composition can comprise a spray which can be sprayed onto surfaces (e.g., in a healthcare setting, such as an electronic device, furniture, walls etc.) to render the surface antimicrobial.

In some embodiments, the composition can comprise a paint (e.g., a latex paint, such as a semi-gloss paint) or other coating composition. Once applied to a surface, the composition can dry to form a coating. The zeolite nanoparticles (which have been rendered hydrophobic) preferentially accumulate at the surface of the coating, rendering the coated surface antimicrobial.

In these embodiments, the composition can further comprise a plurality of polymer particles dispersed in the water. The plurality of polymer particles can comprise a copolymer having a theoretical T_{g} of from −10° C. to 50° C., such as from 10° C. to 45° C., or from 17° C. to 35° C. The copolymer can be derived from one or more ethylenically-unsaturated monomers selected from the group consisting of styrene, butadiene, meth(acrylate) monomers, vinyl acetate, vinyl ester monomers and combinations thereof.

In some embodiments, the copolymer can be an acrylic-based copolymer. For example, the copolymer can be derived from one or more (meth)acrylate monomers; one or more carboxylic acid-containing monomers; and optionally one or more additional ethylenically-unsaturated monomers, excluding monomers (i) and (ii). In some cases, the copolymer can be derived from greater than 80% by weight of one or more (meth)acrylate monomers, based on the total weight of all of the monomers used to form the copolymer. The one or more (meth)acrylate monomers can be selected from the group consisting of methyl methacrylate, butyl acrylate, 2-ethylhexylacrylate, and combinations thereof. The one or more carboxylic acid-containing monomers can be selected from the group consisting of acrylic acid, methacrylic acid, itaconic acid, maleic acid, fumaric acid, and combinations thereof. The composition can further comprise additional standard components, such as a pigment, a filler, a coalescent, a co-solvent, a volatile base, or a combination thereof.

Also provided are antimicrobial compositions that comprise a hydrophobic carrier and a population of zeolite nanoparticles dispersed therein. The zeolite nanoparticles can comprise an effective amount of antimicrobial metal ions to kill or inhibit the growth of a microbe.

The zeolite nanoparticles can be hydrophilically modified. For example, the hydrophilically modified zeolite nanoparticles can comprise a surface that has been modified via association of a hydrophilic capping molecule (e.g., a hydrophilic molecule comprising a cationic moiety, wherein the cationic moiety is electrostatically associated with the surface of the zeolite). The hydrophilically modified zeolite nanoparticles can comprise a surface that is covalently modified via reaction with a hydrophilic alkoxysilane, a hydrophilic halosilane, or a combination thereof.

The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possess a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The zeolite nanoparticles can be present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

In some embodiments, the composition can further comprise a surfactant (e.g., a non-ionic surfactant, such as Triton X, Igepal, Tween, Brij). The surfactant can be present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

In some embodiments, the composition can comprise oil-based paints (e.g., semi-gloss paints, varnish, or stain) or coating compositions. Once applied to a surface, the composition can dry to form a coating. The zeolite nanoparticles (which have been rendered hydrophilic) preferentially accumulate at the surface of the coating, rendering the coated surface antimicrobial.

In some of these embodiments, the hydrophobic carrier can comprise a siccative oil, an alkyd resin, or a combination thereof. The siccative oil can have an iodine number of at least 115 (e.g., an iodine number of from 115 to 180), such as an iodine number of at least 130 (e.g., an iodine number of from 130 to 180). For example, the siccative oil can comprise linseed oil, tung oil, or a combination thereof. The composition can further comprise additional standard components, such as a pigment, a filler, a co-solvent, or a combination thereof.

As mentioned above, these antimicrobial compositions can be applied to surfaces to produce antimicrobial coatings. Accordingly, also provided are methods of producing a coating on a surface that comprise (a) applying to the surface an antimicrobial composition described above; and (b) allowing the composition to dry to produce the coating.

The coating can kill and/or inhibit the growth of a microbe. The microbe can be selected from a bacteria, a fungi, a virus, an algae, or a combination thereof. In some examples, the microbe can be a bacteria selected from Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas fourescens, Nocardia brasiliensis, Nocardia globerula, Acinetobacter genomospecies, Acinetobacter calcoaceticus, Acinetobacter baumannii, Stenotrophomonas maltophlia, Pantoea stewartii ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof. In some examples, the microbe can be a fungi selected from Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof. In some embodiments, the microbe is a coronavirus, such as SARS-CoV-2.

Also provided are dryer sheets comprising a nonwoven substrate; and a transferrable carrier comprising a population of zeolite nanoparticles dispersed therein disposed on the nonwoven substrate. The zeolite nanoparticles released during the clothes drying process comprise an effective amount of antimicrobial metal ions to kill or inhibit the growth of a microbe.

The zeolite nanoparticles can be hydrophobically modified. The modified zeolite nanoparticles can comprise a surface that has been modified via association of a hydrophobic capping molecule. For example, the hydrophobic capping molecule can comprise a hydrophobic molecule comprising a cationic moiety, and wherein the cationic moiety is electrostatically associated with the surface of the zeolite. For example, the capping molecule can comprise an amine defined by Formula I or Formula II below

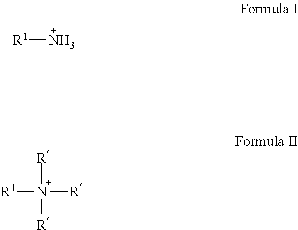

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the modified zeolite nanoparticles can comprise a surface that is covalently modified via reaction with an alkoxysilane, for example an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

In some embodiments, the modified zeolite nanoparticles can comprise a surface that is covalently modified via reaction with an halosilane, for example a halosilane selected from octadecyltrichlorosilane (OTS), hexyltrichlorosilane (HTS), ethyltrichlorosilane (ETS), and combinations thereof.

The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possess a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The transferrable carrier on the drying sheet can comprise, for example, a wax. The zeolite nanoparticles can be present in the transferrable carrier at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm. The transferrable carrier further comprises a fabric softening material, a perfume, a fragrance, an antistatic compound, a soil release agent, an optical brightener, an odor control agent, a fiber lubricant, an antioxidant, a sunscreen, or any combination thereof

When drying wet laundry in a tumble-type dryer in the presence of the sheet, at least a portion of the zeolite nanoparticles can transfer from the nonwoven substrate to the laundry once the temperature within the tumble-type dryer is greater than about 120° F. This can disperse antimicrobial nanoparticles within the laundry, producing a laundered fabric which is antimicrobial.

Accordingly, also provided are methods of applying an antimicrobial agent to a recipient textile which comprise drying the recipient textile in a tumble-type dryer in the present of a dyer sheet described above.

Also provided are textile comprising a woven or nonwoven substrate, and a population of zeolite nanoparticles disposed on the woven or nonwoven substrate. The zeolite nanoparticles can comprise an effective amount of antimicrobial metal ions to kill or inhibit the growth of a microbe.

The woven or nonwoven substrate can comprise, for example, a fabric, garment, bedsheet, linen, surgical gown, surgical drape, wound dressing, medical device, or uniform.

In some embodiments, the woven or nonwoven substrate can comprise fibers formed from cotton, polyester, spandex, rayon, nylon, wool, silk, or a combination thereof. The zeolite nanoparticles can be covalently attached to the woven or nonwoven substrate, non-covalently adsorbed to the woven or nonwoven substrate.

The zeolite nanoparticles are hydrophobically or hydrophilically modified, as described above. The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possesses a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The zeolite nanoparticles are present on the substrate at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

Also provided are hemostatic compositions. Such compositions can comprise a binder; and a population of zeolite nanoparticles dispersed therein. The zeolite nanoparticles can comprise an effective amount of antimicrobial metal ions to kill or inhibit the growth of a microbe.

The zeolite nanoparticles are hydrophobically or hydrophilically modified, as described above. The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possesses a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

In some embodiments, the composition can comprise a first population of zeolite nanoparticles and a second population of zeolite nanoparticles dispersed in the binder, wherein the first population of zeolite nanoparticles comprises an effective amount of silver ions to kill or inhibit the growth of a microbe; and wherein the second population of zeolite nanoparticles comprises an effective amount of calcium ions to enhance blood coagulation upon application of the composition to a wound. In some embodiments, the composition can further comprise an additional population of zeolite nanoparticles dispersed in the binder, wherein the additional population of zeolite nanoparticles comprises an effective amount of zinc ions to kill or inhibit the growth of a microbe. In some embodiments, the composition can further comprise an additional population of zeolite nanoparticles dispersed in the binder, wherein the additional population of zeolite nanoparticles comprises an effective amount of copper ions to kill or inhibit the growth of a microbe.

The binder can be clay-based and may further include fillers (e.g., aluminum sulfate) or thickening agents that facilitate the selective application of the zeolite in various forms (e.g., as a paste, gel, powder, or erodable solid member). Natural clays that may provide suitable bases include, but are not limited to, kaolin, kaolinite, bentonite, montmorillonite, combinations of the foregoing clays, and the like. Modified clays such as polyorganosilcate graft polymers may also provide suitable bases. In some embodiments, the binder can be clay-based, such as kaolin, kaolinite, bentonite, montmorillonite, or a combination thereof.

In some embodiments, the binder can be of an irregularly-shaped granular form having a size distribution determined by sieving ground material with 40 mesh and 16 mesh cut-off screens. Similar clay-based hemostatic compositions are known in the art. As is known in the art, these compositions can be applied to a wound (optionally within a bandage) to rapidly induce clotting. In other embodiments, the binder can be absent, and zeolite alone can be applied to the wound (optionally within a bandage). If desired, the binder can be absent, and the zeolites can be covalently attached to and/or adsorbed to a wound dressing which can be used to induce clotting. Such compositions can be used in trauma situations to rapidly induce clotting in the case of severe bleeding or trauma.

Accordingly, also provided are methods for promoting blood coagulation comprising: applying a wound dressing, covering, or application system to a bleeding area, wherein the wound dressing, covering, or application system comprises a hemostatic composition described herein; and a gauze pad, a multiple layer cover, or a permeable bandage.

Also provided are methods for promoting blood coagulation comprising applying a hemostatic composition described herein to a bleeding area; and applying a gauze pad, a multiple layer cover, or a permeable bandage to a bleeding area.

Also provided are methods of clotting blood flowing from a wound, said method comprising the steps of applying a hemostatic composition described herein to said wound, said composition being capable of producing a controllable blood clotting effect on said wound; and maintaining the composition in contact with said wound for an amount of time sufficient to cause blood flowing from said wound to clot.

In addition to providing antimicrobial benefits, the compositions and articles described herein can also reduce or eliminate odors.

Also provided are polymeric compositions that comprise a polymeric substrate, and a population of zeolite nanoparticles disposed within the polymeric substrate. The zeolite nanoparticles can comprise an effective amount of antimicrobial metal ions to kill or inhibit the growth of a microbe.

The polymeric substrate can comprise any suitable polymeric substrate, such as a synthetic polymer, formed in any suitable form.

The zeolite nanoparticles are hydrophobically or hydrophilically modified, as described above. The zeolite nanoparticles can be porous and the antimicrobial metal ions can be disposed within and/or on a surface of the zeolite nanoparticles. The antimicrobial metal ions can comprise silver, copper, zinc, or a combination thereof. The average particle size of the zeolite nanoparticles can be 100 nm or less (e.g., 80 nm or less). In certain cases, the zeolite nanoparticles have an average particle size of from 10 to 100 nm (e.g., from 20 to 60 nm). The zeolite nanoparticles can possesses a very regular pore structure of molecular dimensions. In some cases, the zeolite nanoparticles can exhibit a monodisperse pore size distribution. In certain embodiments, the zeolite nanoparticles can exhibit an internal pore size of from 2 to 13 angstroms and/or an external pore size of from 10 to 75 angstroms (e.g., from 10 to 50 angstroms) due to packing of the nanoparticles. The zeolite nanoparticles can also possess a high internal surface area. For example, in some embodiments, the zeolite nanoparticles can exhibit an internal surface area of at least 150 m²/g (e.g. at least 200 m²/g, at least 300 m²/g, at least 350 m²/g, or from 300 to 700 m²/g). In some embodiments, the zeolite nanoparticles can have a faujasite structure.

In some embodiments, the antimicrobial metal ions can comprise nanoparticles formed from an antimicrobial metal (e.g., Cu, Zn, Ag, or a combination thereof). The metal nanoparticles can have an average size of 10 nm or less (e.g., from 1 nm to 10 nm or from 1 nm to 5 nm). The amount of metal nanoparticles present in the antimicrobial agents can be 1% by weight or greater, based on the total weight of the zeolite nanoparticles and the metal nanoparticles. In some embodiments, the metal nanoparticles can be present in an amount from 1% to 25% by weight (e.g., from 1% to 20% by weight, from 5% to 25% by weight, from 5% to 20% by weight, from 10% to 25% by weight, from 10% to 20% by weight, or from 15% to 25% by weight), based on the total weight of the zeolite nanoparticles and the metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. The ions may be retained at ion-exchangeable sites of the zeolite nanoparticles. The ions can be present in an amount of 10% or greater (e.g., from 10% up to 100%, from 10% to 95%, from 20% up to 100%, from 30% up to 100%, from 40% up to 100%, or from 50% up to 100%) of the ion exchange capacity of the zeolite nanoparticles.

The zeolite nanoparticles can further comprise an adjuvant. In some embodiments, the adjuvant includes hydrogen ions. The hydrogen ions may be present in an effective amount to reduce the pH of a region (e.g., an aqueous region) in contact with the zeolite nanoparticles. In some embodiments, the adjuvant includes a small molecule antimicrobial agent. In some cases, the small molecule antimicrobial agent is hydrophilic. The small molecule antimicrobial agent can include an antibiotic, an antiseptic, or a disinfectant. The small molecule antimicrobial agent can be present in an amount of from 1% to 20% by weight, based on the total weight of the zeolite nanoparticles and the silver nanoparticle.

The zeolite nanoparticles are present on the substrate at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

### DESCRIPTION OF DRAWINGS

FIG. 1 is a schematic illustration of certain antimicrobial compositions described herein.

FIG. 2 shows high-resolution TEM images of nanozeolite (i) during the synthesis process, (ii) from intermediate steps, and (iii) from the final product. These data show that the particles are always less than 100 nm in size during the entire particle evolution process. The distribution of particle sizes is shown in the histogram. Particle morphology indicates cuboctahedral particles. Average particle size from (iii), the final product, is 31 f 4 nm.

FIG. 3 shows pictures of nanozeolite in water (left) and in cyclohexane (right), demonstrating demonstrates that the nanozeolites, because of their size, can be made into uniform and stable dispersions (without settling) in water and organic solvents.

FIG. 4 is a plot showing data comparing antimicrobial activity towards E. coli. The Y axis displays number of bacterial counts on a log scale; the X-axis displays the materials studied (all concentrations at 100 ppm). Abbreviations: NZ-nanozeolite alone, AgNZ-silver ion exchanged nanozeolite, AgNPNZ-silver nanoparticles in and on nanozeolite, ZnAgNZ-zinc ion and silver ions exchanged into nanozeolite, ZnAgNPNZ-zinc ions exchanged into nanozeolite containing Ag nanoparticles.

FIG. 5 is a plot showing E. coli growth on substrates treated with water-based spray at 1000 ppm zinc ion silver ion nanozeolite (all data with ZnAgNZ at zero counts, so not seen on the graph, ppm concentration refers to the nanozeolite). The water-based spray is only achievable because the nanozeolites can remain suspended in the medium (micron-sized particles and aggregates of nanosized particles will settle down—see FIG. 3). This further enhances the benefits of the discrete nanoparticles described herein.

FIGS. 6A-6B are plots showing the minimum inhibitory concentration (MIC) comparison of NZ samples towards (FIG. 6A) E. coli and (FIG. 6B) MRSA. The best antimicrobial sample is nanozeolite with both zinc ion and silver ions Zn²⁺—Ag⁺—NZ, with MIC of 780 μg/ml towards E Coli and 6.25 μg/ml towards MRSA (methicillin resistant Staphylococcus aureus) (see Table=for comparisons with micron-sized zeolites and colloidal silver nanoparticles). Grey dots indicate very minimal growth and transition between complete growth (black dots) and no growth (white dots), and the concentration at the red dots indicate MIC values.

FIGS. 7A-7B show HRTEM micrographs of E. coli exposed to nanozeolite. (FIG. 7A) Black dots on the surface of the E. coli are the nanozeolite particles surrounding the bacteria (FIG. 7B) Black dots inside the E. coli are particles engulfed by the bacteria.

FIG. 8 is a plot showing the antimicrobial activity towards E. Coli. H—AgNZ is a proton containing silver-ion exchanged nanozeolite sample.

FIG. 9 is a plot showing the antimicrobial activity towards E. Coli. A-ZnAgNZ is a surface derivatized zinc ion and silver-ion containing nanozeolite sample. The surface derivatization was done with 3-aminopropyltriethoxysilane (APTES), which results in positively charged NH₃⁺ groups extending out from the nanozeolite surface.

FIG. 10 is a plot showing the antimicrobial activity of a surface modified with long chain amines (hexadecyl amine, HDA), in which the NH₃⁺ group of a C₁₆ amine binds to the surface of the nanozeolite, and the long chain C₁₆ alkyl group is suspended out and ready to interact with bacteria. A control sample of 1000 ppm HDA-NZ showed E. coli counts of 4.3×10⁴ CFU/ml, whereas HDA-ZnAgNZ (silver ion and zinc ion exchanged zeolite) killed all the bacteria (counts of 0 CFU/ml).

FIG. 11 shows a TEM of silver nanoparticles (bright spots) on a nanozeolite.

FIG. 12A shows a glass plate coating with an aqueous composition comprising antimicrobial zeolite nanoparticles and an optical tracer (e.g., a fluorescent dye) associated with the zeolite nanoparticles. The photographs were obtained under irradiation with UV light. The left image shows the panel before rubbing. The right image shows the panel after the right half of the glass plate was rubbed twenty times. As shown in the right image, rubbing removed the zeolite nanoparticles as illustrated by diminished fluorescence on the right half of the plate.

FIG. 12B shows a glass plate coating with an aqueous composition comprising antimicrobial zeolite nanoparticles, an optical tracer (e.g., a fluorescent dye) associated with the zeolite nanoparticles, and a water-soluble binder polymer. The photographs were obtained under irradiation with UV light. The left image shows the panel before rubbing. The right image shows the panel after the right half of the glass plate was rubbed twenty times. As shown in the right image, rubbing removed some of the zeolite nanoparticles; however, the fluorescence was much greater than observed in FIG. 12A, suggesting that the binder polymer increased the adhesion of the zeolite nanoparticle coating to the glass surface.

FIG. 13 is a plot showing the killing of MRSA on a glass plate coated with a composition that included antimicrobial zeolite nanoparticles, an optical tracer (e.g., a fluorescent dye) associated with the zeolite nanoparticles, and a water-soluble binder polymer.

FIG. 14 illustrates the cage-like structure of a zeolite.

FIG. 15 illustrates transmission electron microscopy of a zeolite on the nanoscale.

FIG. 16 shows E Coli (E) and MRSA (M) tests with ZnAgZN at different concentrations.

FIG. 17 is a schematic representation of a device (e.g., a blood clotting device) described herein.

FIG. 18 is a side view of the device of FIG. 17 illustrating the retaining of blood clotting particles in a mesh container.

FIG. 19 is a side view of a pressure pad incorporating a nanozeolite encapsulated in a mesh container for pressure application to a bleeding wound.

FIG. 20 is a perspective view of a bandage incorporating zeolite nanoparticles in a mesh container for application to a bleeding wound.

FIG. 21 is a side view of a blood clotting device incorporating zeolite nanoparticles retained in a mesh.

FIG. 22 is a side view of one embodiment of the mesh of the device of FIG. 21.

FIG. 23 is a side view of another embodiment of the mesh of the device of FIG. 21.

FIG. 24 is a side view of another embodiment of the mesh of the device of FIG. 21.

FIG. 25 is a side view of a bandage incorporating blood clotting particles retained in a zeolite-impregnated mesh material.

FIG. 26 demonstrates the concept of dryer sheet. Left panel: Control sample. Wet bar rag dried with commercial dryer sheet and then exposed to MRSA (both towel and dryer sheet). Sample placed on agar plate (place), after 24 hours material removed, underneath the material (both bar towel and dryer sheet), MRSA colonies can be observed. If the dryer sheet and bar towel is tapped on the agar plate, further growth of colonies is observed. Right panel: Dryer sheet impregnated with AgZnNZ. Similar experiments as in control, with bar towel and dryer sheet showing fewer colonies (after going through the drying process) of MRSA.

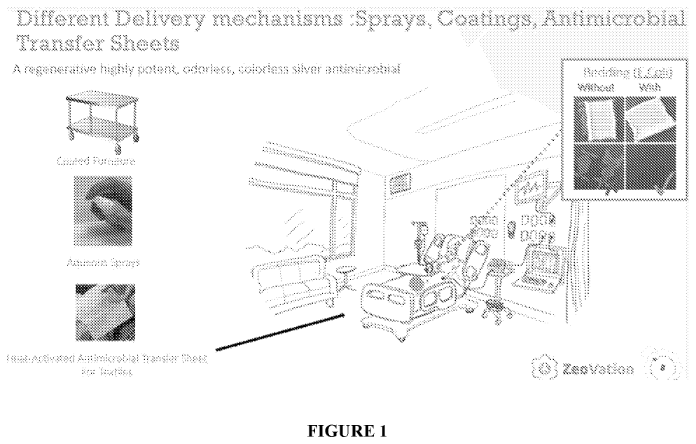

### DETAILED DESCRIPTION

Zeolite Nanoparticles

The zeolite nanoparticles are generally aluminosilicate having a three-dimensionally grown skeleton structure and is generally shown by xM_{2/n}O.Al₂O₃.ySiO₂.zH₂O, wherein M represents an ion-exchangeable metal ion; n corresponds to the valence of the metal; x is a coefficient of the metal oxide; y is a coefficient of silica; and z is the number of water of crystallization. The zeolite nanoparticles can have varying frameworks and differing Si/Al ratios. In some embodiments, the zeolite nanoparticles can comprise zeolite having a faujasite structure. For example, the zeolite nanoparticles can be zeolite X or Y.

The zeolite nanoparticles can have an average particle size of less than 250 nm (e.g., less than 200 nm, less than 150 nm, less than 100 nm, less than 90 nm, less than 80 nm, less than 70 nm, less than 60 nm, less than 50 nm, less than 40 nm, less than 30 nm, or less than 20 nm). In some embodiments, the zeolite nanoparticles can have an average particle size of at least 10 nm (e.g., at least 20 nm, at least 30 nm, at least 40 nm, at least 50 nm, at least 60 nm, at least 70 nm, at least 80 nm, at least 90 nm, at least 100 nm, at least 150 nm, or at least 200 nm).

The zeolite nanoparticles can have an average particle size ranging from any of the minimum values described above to any of the maximum values described above. For example, in some embodiments, the zeolite nanoparticles can have an average particle size of from 10 to 250 nm (e.g., from 10 to 200 nm, from 10 to 150 nm, from 10 to 100 nm, from 20 to 80 nm, or from 20 to 60 nm).

The zeolite nanoparticles can possess a very regular pore structure of molecular dimensions. In some cases, the zeolite nanoparticles can exhibit a monodisperse pore size distribution. As used herein, a monodisperse pore size distribution refers to pore size distributions in which 80% of the distribution (e.g., 85% of the distribution, 90% of the distribution, or 95% of the distribution) lies within 20% of the median pore size (e.g., within 15% of the median pore size, within 10% of the median pore size, or within 5% of the median pore size).

In certain embodiments, the zeolite nanoparticles can exhibit an external pore size of 75 angstroms or less (e.g., 70 angstroms or less, 65 angstroms or less, 60 angstroms or less, 55 angstroms or less, 50 angstroms or less, 45 angstroms or less, 40 angstroms or less, 35 angstroms or less, 30 angstroms or less, 25 angstroms or less, 20 angstroms or less, or 15 angstroms or less). In certain embodiments, the zeolite nanoparticles can exhibit an external pore size of at least 10 angstroms (e.g., at least 15 angstroms, at least 20 angstroms, at least 25 angstroms, at least 30 angstroms, at least 35 angstroms, at least 40 angstroms, at least 45 angstroms, at least 50 angstroms, at least 55 angstroms, at least 60 angstroms, at least 65 angstroms, or at least 70 angstroms).

The zeolite nanoparticles can exhibit an external pore size of from any of the minimum values described above to any of the maximum values described above. For example, in some embodiments, the zeolite nanoparticles can exhibit an external pore size of from 10 to 75 angstroms (e.g., from 10 to 50 angstroms). In certain embodiments, the zeolite nanoparticles can exhibit an internal pore size of 8 angstroms or less (e.g., an internal pore size of from 2 to 8 angstroms).

The zeolite nanoparticles can also possess a high internal surface area. For example, in some embodiments, the zeolite nanoparticles can exhibit an average internal surface area of from 100 to 1,000 m²/g (e.g., from 200 to 1,000 m²/g, from 100 to 800 m²/g, from 200 to 800 m²/g, from 300 to 800 m²/g, from 300 to 700 m²/g, from 100 to 500 m²/g, from 200 to 500 m²/g, or from 400 to 800 m²/g).

The ion-exchange capacities of the zeolite nanoparticles may depend on the silica/aluminum ratio in their formulation. Zeolite types with low silica/aluminum ratios generally exhibit high ion-exchange capacities. In some embodiments, the SiO₂/Al₂O₃ mole ratio in the zeolite nanoparticles is 14 or less (e.g., 13 or less, 12 or less, 11 or less, 10 or less, 9 or less, 8 or less, 7 or less, 6 or less, or 5 or less). In some embodiments, the zeolite nanoparticles can retain a metal ion in an amount as large as or less than an ion-exchange saturation capacity of the zeolite nanoparticles.

Antimicrobial Ions

The zeolite nanoparticles can comprise an effective amount of antimicrobial metal ions to kill or inhibit the growth of a microbe. Suitable antimicrobial metal ions are known in the art, and include silver, copper, zinc, and combinations thereof.

In some embodiments, the antimicrobial metal ions can comprise nanoparticles formed from an antimicrobial metal (e.g., Cu, Zn, Ag, or a combination thereof).

The nanoparticles can have an average particle size of 15 nm or less (e.g., 10 nm or less, 9 nm or less, 8 nm or less, 7 nm or less, 6 nm or less, 5 nm or less, 4 nm or less, 3 nm or less, 2 nm or less, or even 1 nm). In certain embodiments, the nanoparticles can have an average particle size of at least 1 nm (e.g., at least 2 nm, at least 3 nm, at least 4 nm, at least 5 nm, at least 6 nm, at least 7 nm, at least 8 nm, at least 9 nm, or up to 10 nm).

The nanoparticles can have an average particle size ranging from any of the minimum values described above to any of the maximum values described above. For example, in some embodiments, the nanoparticles can have an average particle size of from 1 to 10 nm (e.g., from 1 to 8 nm, or from 1 to 5 nm).

The nanoparticles can be present in an amount of at least 1% by weight (e.g., at least 5% by weight, at least 10% by weight, at least 15% by weight, at least 20% by weight, or at least 25% by weight), based on the total weight of the zeolite nanoparticles and metal nanoparticles. In certain embodiments, the nanoparticles can be present in an amount of 25% by weight or less (e.g., 22% by weight or less, 20% by weight or less, 15% by weight or less, 10% by weight or less, or 5% by weight or less), based on the total weight of the zeolite nanoparticles and metal nanoparticles.

The nanoparticles can be present in an amount ranging from any of the minimum values described above to any of the maximum values described above. For example, in some embodiments, the nanoparticles can be present in an amount from 1% to 25% by weight (e.g., from 5% to 20% by weight, from 5% to 25% by weight, from 10% to 20% by weight, or from 15% to 25% by weight), based on the total weight of the zeolite nanoparticles and metal nanoparticles.

In some embodiments, the antimicrobial metal ions comprise antimicrobial metal ions (copper ions, zinc ions, silver ions, or a combination thereof) retained at ion-exchangeable sites within the zeolite nanoparticles. That is, the ion-exchangeable ions such as sodium ions, calcium ions, potassium ions, magnesium ions and/or iron ions in the zeolite nanoparticles can be partially or wholly replaced with the antimicrobial metal ions.

The antimicrobial metal ions can be present in an amount as large as or less than the ion-exchange saturation capacity of the zeolite nanoparticles. In some embodiments, the zeolite nanoparticles retain antimicrobial metal ions in an amount of 10% or greater, 15% or greater, 20% or greater, 25% or greater, 30% or greater, 40% or greater, 50% or greater, 60% or greater, 75% or greater, 80% or greater, 90% or greater, 95% or greater, or up to 100%, of the ion exchange capacity of the zeolite nanoparticles. In some embodiments, the zeolite nanoparticles can retain the antimicrobial metal ions in an amount of 100% or less, 95% or less, 90% or less, 85% or less, 75% or less, 50% or less, 40% or less, or 25% or less, of the ion exchange capacity of the zeolite nanoparticles.

The antimicrobial metal ions can be present in an amount ranging from any of the minimum values described above to any of the maximum values described above. For example, in some embodiments, the antimicrobial metal ions can be retained in an amount from 10% up to 100% by weight (e.g., from 20% up to 100%, from 30% up to 100%, from 40% up to 100%, or from 50% up to 100%), of the ion exchange capacity of the zeolite nanoparticles.

In some embodiments, the zeolite nanoparticles can further comprise an additional bioactive metal ion (e.g. calcium ions) retained at ion-exchangeable sites within the zeolite nanoparticles. That is, the ion-exchangeable ions such as sodium ions, calcium ions, potassium ions, magnesium ions and/or iron ions in the zeolite nanoparticles can be partially or wholly replaced with the calcium ions. The calcium can induce clotting in the case of hemostatic compositions.

The additional bioactive metal ions can be present in an amount as large as or less than the ion-exchange saturation capacity of the zeolite nanoparticles. In some embodiments, the zeolite nanoparticles retain antimicrobial metal ions in an amount of 10% or greater, 15% or greater, 20% or greater, 25% or greater, 30% or greater, 40% or greater, 50% or greater, 60% or greater, 75% or greater, 80% or greater, 90% or greater, 95% or greater, or up to 100%, of the ion exchange capacity of the zeolite nanoparticles. In some embodiments, the zeolite nanoparticles can retain the antimicrobial metal ions in an amount of 100% or less, 95% or less, 90% or less, 85% or less, 75% or less, 50% or less, 40% or less, or 25% or less, of the ion exchange capacity of the zeolite nanoparticles.

The antimicrobial metal ions can be present in an amount ranging from any of the minimum values described above to any of the maximum values described above. For example, in some embodiments, the antimicrobial metal ions can be retained in an amount from 10% up to 100% by weight (e.g., from 20% up to 100%, from 30% up to 100%, from 40% up to 100%, or from 50% up to 100/), of the ion exchange capacity of the zeolite nanoparticles.

Surface Modification

The zeolite nanoparticles can be hydrophobically or hydrophilically modified.

In some embodiments, the zeolite nanoparticles can be hydrophobically modified. The modified zeolite nanoparticles can comprise a surface that has been modified via association of a hydrophobic capping molecule. For example, the hydrophobic capping molecule can comprise a hydrophobic molecule comprising a cationic moiety, and wherein the cationic moiety is electrostatically associated with the surface of the zeolite. For example, the capping molecule can comprise an amine defined by Formula I or Formula II below

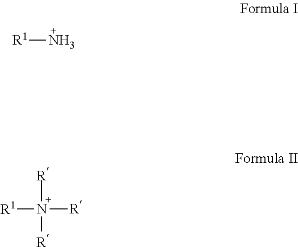

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the modified zeolite nanoparticles can comprise a surface that is covalently modified via reaction with an alkoxysilane, for example an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

In some embodiments, the modified zeolite nanoparticles can comprise a surface that is covalently modified via reaction with an halosilane, for example a halosilane selected from octadecyltrichlorosilane (OTS), hexyltrichlorosilane (HTS), ethyltrichlorosilane (ETS), and combinations thereof.

Adjuvants

The zeolite nanoparticles described herein can comprise an adjuvant. The term “adjuvant” as described herein refers to a substance added to or co-formulated with the compositions described herein to enhance, induce, elicit, and/or modulate the antimicrobial activity of silver when contacted to a microbe.

In some embodiments, the adjuvant comprises antimicrobial metal ions. The antimicrobial metal ions can include a metal selected from copper ions, zinc ions, mercury ions, lead ions, tin ions, bismuth ions, cadmium ions, chromium ions, antimony ions, arsenic ions, or thallium ions. In some examples, the adjuvant can include copper ions, zinc ions, or a combination thereof. The antimicrobial metal ions can be present in an amount of 100% or less, 95% or less, 90% or less, 85% or less, 75% or less, 70% or less, 60% or less, 50% or less, 40% or less, 30% or less, 25% or less, 20% or less, 15% or less, 10% or less, of the ion exchange capacity of the zeolite nanoparticles.

In some embodiments, the adjuvant comprises hydrogen ions. The hydrogen ions can be present in an amount to reduce the pH of an aqueous region in contact with the zeolite nanoparticles.

In some embodiments, the adjuvant comprises a small molecule antimicrobial agent. “Small Molecule”, as used herein, refers to a molecule, such as an organic compound, with a molecular weight of less than about 2,000 Daltons (e.g., less than about 1,500 Daltons, less than about 1,000 Daltons, or less than about 800 Daltons). The small molecule antimicrobial agent can be selected from an antibacterial agent, an antiviral agent, and/or an antifungal agent.

Suitable examples of small molecule antimicrobial agent include antibiotics, disinfectant, antiseptics, or a combination thereof. In certain embodiments, the small molecule antimicrobial agent can include a hydrophilic small molecule.

Representative examples of small molecule antimicrobial agents include, for example, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, and triclocarban.

Examples of antibacterials include, for example, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, and xibornol.

Examples of antifungals include, for example, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb and ziram.

Examples of antivirals include, for example, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol and ZMapp.

The small molecule antimicrobial agent can be present in an amount from 0.1% to 20% by weight (e.g., from 0.1% to 20% by weight, from 0.1% to 15% by weight, from 0.1% to 10⁰/a by weight, or from 0.1% to 5% by weight), based on the total weight of the zeolite nanoparticles and silver.

Targeting Agents

The zeolite nanoparticles can also include a microbial targeting agent. The microbial targeting agent can be covalently linked to the zeolite nanoparticles. Some microbes are known to have a negative charge density on their surface. Therefore, in some embodiments, the microbial targeting agent can comprise a cationic group or a cationic precursor. In some embodiments, the microbial targeting agent can comprise an amine containing group. The amine containing group can include an alkyl amine such as a C₁-C₁₂ alkyl amine.

The microbial targeting agent can be present in an amount from 0.1% to 20% by weight (e.g., from 1% to 20% by weight, from 1% to 15% by weight, from 1% to 10% by weight, or from 0.1% to 5% by weight), based on the total weight of the zeolite nanoparticles and silver.

In some embodiments of the antimicrobial agents disclosed herein, the antimicrobial agent can include zeolite nanoparticles, wherein the zeolite nanoparticles further comprise silver nanoparticles disposed on and/or within the zeolite nanoparticles. In other embodiments of the antimicrobial agents disclosed herein, the antimicrobial agent can include zeolite nanoparticles, wherein the zeolite nanoparticles further comprise silver nanoparticles disposed on and/or within the zeolite nanoparticles and antimicrobial metal ions retained at ion-exchangeable sites within the zeolite nanoparticles. In further embodiments of the antimicrobial agents disclosed herein, the antimicrobial agent can include zeolite nanoparticles, wherein the zeolite nanoparticles further comprise silver nanoparticles disposed on and/or within the zeolite nanoparticles and wherein a surface of the zeolite nanoparticles is functionalized with a microbial targeting agent. In still further embodiments of the antimicrobial agents disclosed herein, the antimicrobial agent can include zeolite nanoparticles, wherein the zeolite nanoparticles further comprise silver nanoparticles disposed on and/or within the zeolite nanoparticles and a small molecule antimicrobial agent adsorbed on and/or within the zeolite nanoparticle.

Optical Tracers

In some embodiments, the composition can further comprise an optical tracer associated with the zeolite nanoparticles. In some cases, the optical tracer can be covalently bound to the zeolite nanoparticles. In some cases, the optical tracer can be non-covalently associated with the zeolite nanoparticles.

By way of example, in some embodiments, the optical tracer can be adsorbs to the zeolite nanoparticles. In these embodiments, the high external surface area of the zeolite can adsorb a dye. In other embodiments, electrostatic attachment can be used to associate the optical tracer with the zeolite nanoparticles. For example, cationic dyes (e.g. Rhodamine 6G) can be electrostatically attached to negatively charged zeolite surfaces. Alternatively, dyes can be functionalized to introduce a cationic moiety, allowing them to be electrostatically attached to negatively charged zeolite surfaces. An example is illustrated for AMCA in the scheme below.

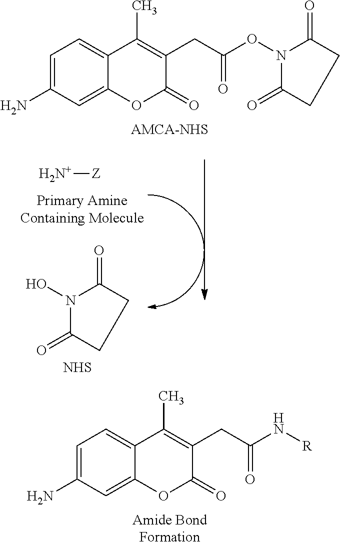

In some embodiments, the optical tracer can be covalently attached to the surface of the zeolite nanoparticles. This can be readily accomplished through reaction with surface hydroxyl groups of zeolite as shown in the scheme below (where X represents a fluorophore)

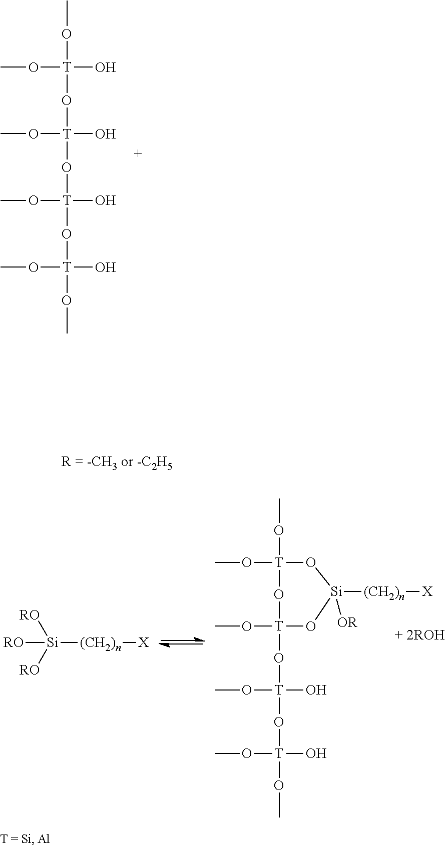

The fluorophore can be selected to possess photophysical properties which facilitate the observation and/or analysis of the spectroscopic properties of the fluorophore. For example, in certain embodiments, the fluorophore possesses a fluorescence quantum yield that facilitates observation and measurement of the chemosensing agent's fluorescence. In some cases, the fluorophore possesses a quantum yield of at least 0.10, 0.15, 0.20, 0.25, 0.30, 0.35, 0.40, 0.45, 0.50, 0.55, 0.60, 0.65, 0.70, 0.75, 0.80, 0.85, or 0.90 in aqueous solution.

In some cases, the chemosensing agent is designed for sensing in biological samples. In biological samples, background fluorescence from cells, tissues and biological fluids (referred to as autofluorescence) can complicate analysis of the fluorescence of the chemosensing agent. In some cases, the fluorophore does not possess an emission maximum in a spectral region which substantially overlaps with the autofluorescence of biological samples. In certain embodiments, the fluorophore possesses an emission maximum greater than 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, Or 700 nm in aqueous solution. In certain embodiments, the fluorophore possesses an emission maximum in aqueous solution between 430 nm and 700 nm, more preferably between 450 nm and 700 nm, most preferably between 480 nm and 700 nm. In some embodiments, the fluorophore possesses an emission maximum in aqueous solution between 430 nm and 1200 nm, more preferably between 450 nm and 1200 nm, most preferably between 480 nm and 1200 nm.

In preferred embodiments, the fluorophore is selected to possess the photophysical properties, including fluorescence quantum yield and emission maxima, desired for a particular sensing application. In some embodiments, the fluorophore possesses a high quantum yield and emits at a long wavelength. In a particular embodiment, the fluorophore possesses an emission maximum greater than 450 nm and a quantum yield of greater than 0.10 in aqueous solution.

Any suitable fluorophore may be incorporated into the chemosensing agents described above. Fluorophores useful in chemosensing agents typically contain an extended conjugation path (e.g., alternating single and double bonds) over which pi electrons are delocalized. The fluorophore can be aromatic, meaning it contains one or more aromatic rings, or non-aromatic (e.g., a linear structure). In preferred embodiments, the fluorophore contains one or more aromatic rings.

In some embodiments, the fluorophore is an organic or organometallic small molecule. Suitable small molecule fluorophores are known in the art, and include, but are not limited to, xanthene and xanthene derivatives, such as fluorescein or a fluorescein derivative, rhodamine, Oregon green, eosin, Texas red, and Cal Fluor dyes; cyanine and cyanine derivatives, such as indocarbocyanine, oxacarbocyanine, thiacarbocyanine, merocyanine, and Quasar dyes; naphthalene derivatives, such as dansyl and prodan derivatives and naphthalimide and naphthalimide derivatives; coumarin and derivatives thereof, oxadiazole derivatives, such as pyridyloxazole, nitrobenzoxadiazole and benzoxadiazole; pyrene derivatives, such as cascade blue; oxazine derivatives, such as Nile red, Nile blue, cresyl violet, and oxazine 170; acridine derivatives; such as proflavin, acridine orange, and acridine yellow; arylmethine derivatives, such as auramine, crystal violet, and malachite green; tetrapyrrole derivatives, such as porphin, phtalocyanine, and bilirubin; fluorene derivatives; CF® dye (available from Biotium); BODIPY® (available from Invitrogen); Alexa Fluor® (available from Invitrogen); DyLight Fluor® (available from Thermo Scientific); Atto® and Tracy® available from Sigma Aldrich; and FluoProbes® (available from Interchim). Other suitable fluorophores include those described in Lakowicz, J. R. “Principles of Fluorescence Spectroscopy”, 2^{nd} Ed., Plenum Press, New York, 1999.

Suitable fluorophores can also include macromolecules, such as conjugated polymers. In some embodiments, the fluorophore is a conjugated polymer, such as a poly(arylene ethynylene), containing one or more sidechains that contain reactive functional groups.

Other suitable optical tracers include semiconductor nanocrystals and lanthanide chelates. A wide variety of fluorescent semiconductor nanocrystals (“SCNCs”) are known in the art; methods of producing and utilizing semiconductor nanocrystals are described in: PCT Publ. No. WO 99/26299 published May 27, 1999, inventors Bawendi et al.; U.S. Pat. No. 5,990,479 issued Nov. 23, 1999 to Weiss et al.; and Bruchez et al., Science 281:2013, 1998. Semiconductor nanocrystals can be obtained with very narrow emission bands with well-defined peak emission wavelengths.

In some cases, the optical tracer can comprise a fluorophore, such as a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.

Methods of Making

Methods of making the antimicrobial zeolites are also disclosed. Methods of making zeolite nanoparticles are described in PCT/2015/060681, which is incorporated herein by reference in its entirety. Briefly, the method can include (a) heating a first mixture comprising a silicon source, an aluminum source, a base, an organic agent, and a first solvent to produce a first population of zeolite nanoparticles dispersed in a first supernatant; (b) separating the first population of zeolite nanoparticles from the first supernatant; (c) adding a base to the first supernatant to form a second mixture; (d) heating the second mixture to produce a second population of zeolite nanoparticles dispersed in a second supernatant; and (e) separating the second population of zeolite nanoparticles from the second supernatant. The first population of zeolite nanoparticles and the second population of zeolite nanoparticles prepared by the methods described herein can each have an average particle size of 250 nm or less, such as 100 nm or less.

In certain embodiments, the methods of making the zeolite nanoparticles can include mixing a silicon source, an aluminum source, a base, an organic agent, and a first solvent to form a first mixture. The silicon and/or aluminum source can include any suitable compound that will hydrolyze to provide silicon and/or aluminum to form the framework of the zeolite nanoparticles. For example, the silicon source can include tetraethylorthosilane (TEOS), colloidal or fumed silica (amorphous silica such as Ludox LS30), disodium metasilicate, or combinations thereof. The aluminum source can include aluminum hydroxide, aluminum isopropoxide, sodium aluminate, aluminum sulfate, or combinations thereof. The organic agent can be a porous material that can serve as the structure around which an alumino-silicate nanoparticles can form. For example, the organic agent can be any suitable organic base. Examples of organic agents can include tetrapropyl ammonium hydroxide (TPAOH), tetramethyl ammonium hydroxide (TMAOH), tetramethyl ammonium bromide, and tetrapropyl ammonium bromide. The base can include transition metal oxides and hydroxides, alkali metal oxides and hydroxides, alkaline earth metal oxides and hydroxides. For example, the base can include sodium hydroxide or potassium hydroxide. The first solvent can include water.

In some embodiments, the first mixture can comprise water, sodium hydroxide, colloidal silica, tetramethyl ammonium hydroxide, aluminum isopropoxide, and tetramethylammonium bromide. In some embodiments, the first mixture can comprise water, sodium hydroxide, tetraethylorthosilane, and tetrapropyl ammonium hydroxide. In some embodiments, the first mixture can comprise water, tetraethylorthosilane, sodium hydroxide, tetramethyl ammonium hydroxide, and aluminum isopropoxide. In some embodiments, the first mixture can comprise water, sodium hydroxide, tetrapropyl ammonium hydroxide, silicon, and ethanol.

The amount of silicon source present in the first mixture can be from 1.7 mol % to 5.2 mol % (e.g., from 3.1 mol % to 3.8 mol %) of the components used to form the first mixture. The amount of aluminum source present in the first mixture can be from 0.01 mol % to 2 mol % (e.g., from 0.02 mol % to 1 mol %) of the components used to form the first mixture. The amount of organic agent present in the first mixture can be from 0.1 mol % to 5 mol % (e.g., from 0.6 mol % to 0.3 mol %) of the components used to form the first mixture. The amount of base present in the first mixture can be from 0.001 mol % to 0.1% mol % (e.g., from 0.0001 mol % to 0.05 mol %) of the components used to form the first mixture. The amount of solvent present in the mixture can be from 90 mol % to 99 mol % (e.g., from 95 mol % to 99 mol %) of the components used to form the first mixture.

In an exemplary method, the silicon source, aluminum source, base, organic agent, and solvent can be combined in a suitable ratio to form a first mixture comprising 0.048 Na₂O:2.40 (TMA)₂O(2OH):1.2 (TMA)₂O(2Br): 4.35 SiO₂:1.0 Al₂O₃:249 H₂O, after hydrolysis.

In another exemplary method of preparing zeolite nanoparticles, Ludox HS-30 and tetramethylammonium hydroxide can be mixed at room temperature to produce a silicon source. Aluminum isopropoxide can be dissolved in water and tetramethylammonium hydroxide. The resulting mixture can be heated followed by addition of tetramethylammonium bromide, thereby forming the aluminum source. The silicon source and aluminum source can be mixed and aged at room temperature with stirring for about three days. The aged mixture can be heated with stirring for about four days. The reacted mixture can be centrifuged to produce zeolite Y nanoparticles and a supernatant. The supernatant can be mixed with sodium hydroxide, aged overnight, and refluxed for about 3 hours to produce a second batch of zeolite Y nanoparticles and a second supernatant. The second batch of zeolite Y nanoparticles can be separated from the supernatant. The addition of sodium hydroxide, aging, heating, and separating the nanoparticles from the supernatant can define one cycle. The cycle can then be repeated eight times.

In a further exemplary method of preparing zeolite nanoparticles, Ludox HS-30 and tetramethylammonium hydroxide can be mixed at room temperature to produce a silicon source. Aluminum isopropoxide can be dissolved in water and tetramethylammonium hydroxide. The resulting mixture can be heated to form a solution followed by addition of tetramethylammonium bromide resulting in the aluminum source. The silicon source and aluminum source can be mixed and aged at room temperature with stirring for about three days. The aged mixture can be heated with stirring for about four days. The reacted mixture can be centrifuged to produce zeolite Y nanoparticles and a supernatant. The supernatant can be mixed with sodium hydroxide, refluxed, and concentrated by removing water (by condensation) for about 30 minutes during reflux. The resulting concentrated solution can be refluxed for an additional 30 minutes. Sodium hydroxide can be dissolved in the condensed water which can be used to dilute the concentrated solution. The water can be added to the concentrated solution over about 30 minutes. The 90 minutes process can define one cycle. The cycle can be repeated for six times (9 hours) to form zeolite Y nanoparticles.

Methods of incorporating silver in the zeolite nanoparticles are describes in J. Phys. Chem. C 2014, 118, 28580-28591, which is incorporated by reference herein. Briefly, a colloidal dispersion of the zeolite nanoparticles can be ion exchanged first with a sodium salt, such as sodium nitrate and then with a silver salt, such as silver nitrate to form zeolite nanoparticles comprising silver ions. The silver ions in the zeolite nanoparticles can be reduced to form silver nanoparticles. In particular, the silver-exchanged zeolite dispersion formed can be reduced using a reducing agent. Preferably, the reducing agent is a weak reducing agent such as resorcinol. By removing the reducing agent at any stage of the reduction, stable silver nanoparticles on zeolite nanoparticles can be isolated. The properties of the zeolite nanoparticles comprising silver can be characterized with optical spectroscopy (e.g. surface-enhanced Raman measurements) and transmission electron microscopy.

Binder Polymers

The compositions described herein can include a binder polymer. In some embodiments, the binder polymer can be a water soluble polymer. “Water-soluble” as used herein refers to a material that is miscible in water. In other words, a material that is capable of forming a stable (does not separate for greater than 5 minutes after forming the homogeneous solution) homogeneous solution with water at ambient conditions.

The binder polymer may be synthetic or natural original and may be chemically and/or physically modified. In one example, the binder polymer can exhibit a weight average molecular weight of at least 10,000 g/mol and/or at least 20,000 g/mol and/or at least 40,000 g/mol and/or at least 80,000 g/mol and/or at least 100,000 g/mol and/or at least 1,000,000 g/mol and/or at least 3,000,000 g/mol and/or at least 10,000,000 g/mol and/or at least 20,000,000 g/mol and/or to about 40,000,000 g/mol and/or to about 30,000,000 g/mol.

In some examples, the binder polymer can include water-soluble hydroxyl polymers, water-soluble thermoplastic polymers, water-soluble biodegradable polymers, water-soluble non-biodegradable polymers and mixtures thereof. In one example, the water-soluble polymer comprises polyvinyl alcohol. In another example, the water-soluble polymer comprises starch. In yet another example, the water-soluble polymer comprises polyvinyl alcohol and starch. In yet another example, the water-soluble polymer comprises polyethylene glycol.

Non-limiting examples of water-soluble hydroxyl polymers in accordance with the present invention include polyols, such as polyvinyl alcohol, polyvinyl alcohol derivatives, polyvinyl alcohol copolymers, starch, starch derivatives, starch copolymers, chitosan, chitosan derivatives, chitosan copolymers, cellulose derivatives such as cellulose ether and ester derivatives, cellulose copolymers, hemicellulose, hemicellulose derivatives, hemicellulose copolymers, gums, arabinans, galactans, proteins and various other polysaccharides and mixtures thereof.

In one example, a water-soluble hydroxyl polymer comprises a polysaccharide.

“Polysaccharides” as used herein means natural polysaccharides and polysaccharide derivatives and/or modified polysaccharides. Suitable water-soluble polysaccharides include, but are not limited to, starches, starch derivatives, chitosan, chitosan derivatives, cellulose derivatives, hemicellulose, hemicellulose derivatives, gums, arabinans, galactans and mixtures thereof. The water-soluble polysaccharide may exhibit a weight average molecular weight of from about 10,000 to about 40,000,000 g/mol and/or greater than 100,000 g/mol and/or greater than 1,000,000 g/mol and/or greater than 3,000,000 g/mol and/or greater than 3,000,000 to about 40,000,000 g/mol.

The water-soluble polysaccharides may comprise non-cellulose and/or non-cellulose derivative and/or non-cellulose copolymer water-soluble polysaccharides. Such non-cellulose water-soluble polysaccharides may be selected from the group consisting of: starches, starch derivatives, chitosan, chitosan derivatives, hemicellulose, hemicellulose derivatives, gums, arabinans, galactans and mixtures thereof.

In another example, a water-soluble hydroxyl polymer comprises a non-thermoplastic polymer.

The water-soluble hydroxyl polymer may have a weight average molecular weight of from about 10,000 g/mol to about 40,000,000 g/mol and/or greater than 100,000 g/mol and/or greater than 1,000,000 g/mol and/or greater than 3,000,000 g/mol and/or greater than 3,000,000 g/mol to about 40,000,000 g/mol. Higher and lower molecular weight water-soluble hydroxyl polymers may be used in combination with hydroxyl polymers having a certain desired weight average molecular weight.

Well known modifications of water-soluble hydroxyl polymers, such as natural starches, include chemical modifications and/or enzymatic modifications. For example, natural starch can be acid-thinned, hydroxy-ethylated, hydroxy-propylated, and/or oxidized. In addition, the water-soluble hydroxyl polymer may comprise dent corn starch.

Naturally occurring starch is generally a mixture of linear amylose and branched amylopectin polymer of D-glucose units. The amylose is a substantially linear polymer of D-glucose units joined by (1,4)-α-D links. The amylopectin is a highly branched polymer of D-glucose units joined by (1,4)-α-D links and (1,6)-α-D links at the branch points. Naturally occurring starch typically contains relatively high levels of amylopectin, for example, corn starch (64-80% amylopectin), waxy maize (93-100% amylopectin), rice (83-84% amylopectin), potato (about 78% amylopectin), and wheat (73-83% amylopectin). Though all starches are potentially useful herein, the present invention is most commonly practiced with high amylopectin natural starches derived from agricultural sources, which offer the advantages of being abundant in supply, easily replenishable and inexpensive.

As used herein, “starch” includes any naturally occurring unmodified starches, modified starches, synthetic starches and mixtures thereof, as well as mixtures of the amylose or amylopectin fractions; the starch may be modified by physical, chemical, or biological processes, or combinations thereof. The choice of unmodified or modified starch for the present invention may depend on the end product desired. In one embodiment of the present invention, the starch or starch mixture useful in the present invention has an amylopectin content from about 20% to about 100%, more typically from about 40% to about 90%, even more typically from about 60% to about 85% by weight of the starch or mixtures thereof.

Suitable naturally occurring starches can include, but are not limited to, corn starch, potato starch, sweet potato starch, wheat starch, sago palm starch, tapioca starch, rice starch, soybean starch, arrow root starch, amioca starch, bracken starch, lotus starch, waxy maize starch, and high amylose corn starch. Naturally occurring starches particularly, corn starch and wheat starch, are the preferred starch polymers due to their economy and availability.

Polyvinyl alcohols herein can be grafted with other monomers to modify its properties. A wide range of monomers has been successfully grafted to polyvinyl alcohol. Non-limiting examples of such monomers include vinyl acetate, styrene, acrylamide, acrylic acid, 2-hydroxyethyl methacrylate, acrylonitrile, 1,3-butadiene, methyl methacrylate, methacrylic acid, maleic acid, itaconic acid, sodium vinylsulfonate, sodium allylsulfonate, sodium methylallyl sulfonate, sodium phenylallylether sulfonate, sodium phenylmethallylether sulfonate, 2-acrylamido-methyl propane sulfonic acid (AMPS), vinylidene chloride, vinyl chloride, vinyl amine and a variety of acrylate esters.

In one example, the water-soluble hydroxyl polymer is selected from the group consisting of: polyvinyl alcohols, hydroxymethylcelluloses, hydroxyethylcelluloses, hydroxypropylmethylcelluloses and mixtures thereof. A non-limiting example of a suitable polyvinyl alcohol includes those commercially available from Sekisui Specialty Chemicals America, LLC (Dallas, Tex.) under the CELVOL® trade name. A non-limiting example of a suitable hydroxypropylmethylcellulose includes those commercially available from the Dow Chemical Company (Midland, Mich.) under the METHOCEL® trade name including combinations with above mentioned hydroxypropylmethylcelluloses.

Non-limiting examples of suitable water-soluble thermoplastic polymers include thermoplastic starch and/or starch derivatives, polylactic acid, polyhydroxyalkanoate, polycaprolactone, polyesteramides and certain polyesters, and mixtures thereof.

The water-soluble thermoplastic polymers may be hydrophilic or hydrophobic. The water-soluble thermoplastic polymers may be surface treated and/or internally treated to change the inherent hydrophilic or hydrophobic properties of the thermoplastic polymer.

The water-soluble thermoplastic polymers may comprise biodegradable polymers.

Any suitable weight average molecular weight for the thermoplastic polymers may be used. For example, the weight average molecular weight for a thermoplastic polymer is greater than about 10,000 g/mol and/or greater than about 40,000 g/mol and/or greater than about 50,000 g/mol and/or less than about 500,000 g/mol and/or less than about 400,000 g/mol and/or less than about 200,000 g/mol.

Methods of Using

As used herein, “inhibit” or other forms of the word, such as “inhibiting” or “inhibition,” refers to lowering of an event or characteristic (e.g., microbe population/infection). It is understood that the inhibition is typically in relation to some standard or expected value. For example, “inhibiting the growth of microbes” means reducing the growth of a microbe relative to a standard or a control.

As described herein, the term “microbes” includes, for example, bacteria, virus, algae, and fungi. In some embodiments, the methods described herein can be used to kill, inhibit, control or prevent microbes such as Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas fourescens, Nocardia brasiliensis, Nocardia globerula, Acinetobacter genomospecies, Acinetobacter calcoaceticus, Acinetobacter baumannii, Stenotrophomonas maltophlia, Pantoea stewartii ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp., Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof. In some embodiments, the microbe is a coronavirus, such as SARS-CoV-2.

The methods and compositions as described herein are useful for medical devices and wound dressing coatings. The medical devices or bandages can be wholly or partially coated with a composition as described herein. In some embodiments, the compositions can be formulated with a wound dressing, coated on a bandage or the exterior surface of a medical device. Exemplary medical devices can include, suture thread, wound closure tape, catheters, tubes, stents, atheroscopic balloons, pace makers, replacement joints (e.g., hip, knee), valves, chips (e.g., information storage media, computer chip, computer-readable media), etc.

The methods and compositions as described herein are also useful in other coatings such as wall coatings (e.g., paints, varnishes, etc.).

Wound Dressings

Also disclosed herein are devices and methods for delivering the zeolite nanoparticles to wounds to promote the clotting of blood and the dressing of the wounds. The devices generally comprise expedients or apparatuses that can be applied to bleeding wounds such that the zeolite nanoparticles contact the tissue of the wound to minimize or stop blood flow of blood by absorbing at least portions of the liquid phases of the blood, thereby promoting clotting. One apparatus comprises a receptacle for retaining zeolite nanoparticles in particulate form. At least a portion of the receptacle is defined by a mesh having openings therein, and at least a portion of the particulate zeolite particles (which can comprise zeolite nanoparticles aggregated and/or combined with a binder polymer or clay-based binder) is in direct contact with blood through the openings. As used herein, the terms “particle” and “particulate” are intended to refer to balls, beads, pellets, rods, granules, polymorphous shapes, and combinations of the foregoing.

In embodiments, the zeolite nanoparticles may be combined with a synthetic polymer gel, cellulosic material, porous silica gel, porous glass, alumina, hydroxyapatite, calcium silicate, zirconia, clay-based material, or the like to form zeolite particles. Exemplary synthetic polymers include, but are not limited to, styrene-divinylbenzene copolymer, cross-linked polyvinyl alcohol, cross-linked polyacrylate, cross-linked vinyl ether-maleic anhydride copolymer, cross-linked styrene-maleic anhydride copolymer or cross-linked polyamide, and combinations thereof.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), bacteriostatics, compounds containing silver ions, and the like. Other materials that can be incorporated to provide additional hemostatic functions include ascorbic acid, tranexamic acid, rutin, and thrombin. Botanical agents having desirable effects on the wound site may also be added.

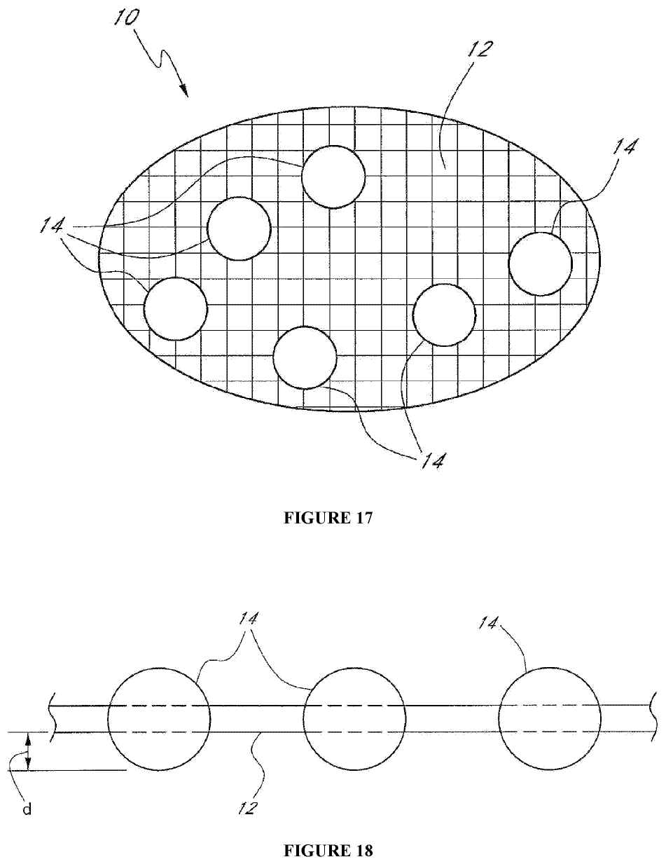

In one embodiment, a device for facilitating the clotting of blood directly at a wound site is shown with reference to FIG. 17. The device is a permeable pouch that allows liquid to enter to contact blood clotting nanozeolite retained therein. Sealed packaging (not shown) provides a sterile environment for storing the device until it can be used. The device, which is shown generally at 10 and is hereinafter referred to as “pouch 10,” comprises a screen or mesh 12 and zeolite particles 14 retained therein by the screen or mesh. The mesh 12 is closed on all sides and defines openings that are capable of retaining the zeolite particles 14 therein while allowing liquid to flow through. As illustrated, the mesh 12 is shown as being flattened out, and only a few zeolite particles 14 are shown.

The zeolite particles 14 can be substantially spherical or irregular in shape (e.g., balls, beads, pellets, or the like) and about 0.2 millimeters (mm) to about 10 mm in diameter, preferably about 1 mm to about 7 mm in diameter, and more preferably about 2 mm to about 5 mm in diameter. In any embodiment (balls, beads, pellets, etc.), less particle surface area is available to be contacted by blood as the particle size is increased. Therefore, the rate of clotting can be controlled by varying the particle size. Furthermore, the adsorption of moisture (which also has an effect on the exothermic effects of the zeolite) can also be controlled. The particles can be formed from fractal-like aggregated zeolite nanoparticles, zeolite nanoparticles in combination with a binder or other additive (e.g., clay), or any combination thereof.

The mesh 12 is defined by interconnected strands, filaments, or strips of material. The strands, filaments, or strips can be interconnected in any one or a combination of manners including, but not limited to, being woven into a gauze, intertwined, integrally-formed, and the like. Preferably, the interconnection is such that the mesh can flex while substantially maintaining the dimensions of the openings defined thereby. The material from which the strands, filaments or strips are fabricated may be a polymer (e.g., nylon, polyethylene, polypropylene, polyester, or the like), metal, fiberglass, or an organic substance (e.g., cotton, wool, silk, or the like).

Referring now to FIG. 18, the openings defined by the mesh 12 are dimensioned to retain the zeolite particles 14 but to accommodate the flow of blood therethrough. Because the mesh 12 may be pulled tight around the zeolite particles 14, the particles may extend through the openings by a distance d. If the zeolite particles 14 extend through the openings, the particles are able to directly contact tissue to which the pouch 10 is applied. Thus, blood emanating from the tissue immediately contacts the zeolite particles 14, and the water phase thereof is wicked into the zeolite material, thereby facilitating the clotting of the blood. However, it is not a requirement that the zeolite particles protrude through the mesh.

To apply the pouch 10 to a bleeding wound, the pouch is removed from the packaging and placed on the bleeding wound. The zeolite particles 14 in the mesh 12 contact the tissue of the wound and/or the blood, and at least a portion of the liquid phase of the blood is adsorbed by the zeolite material, thereby promoting the clotting of the blood.

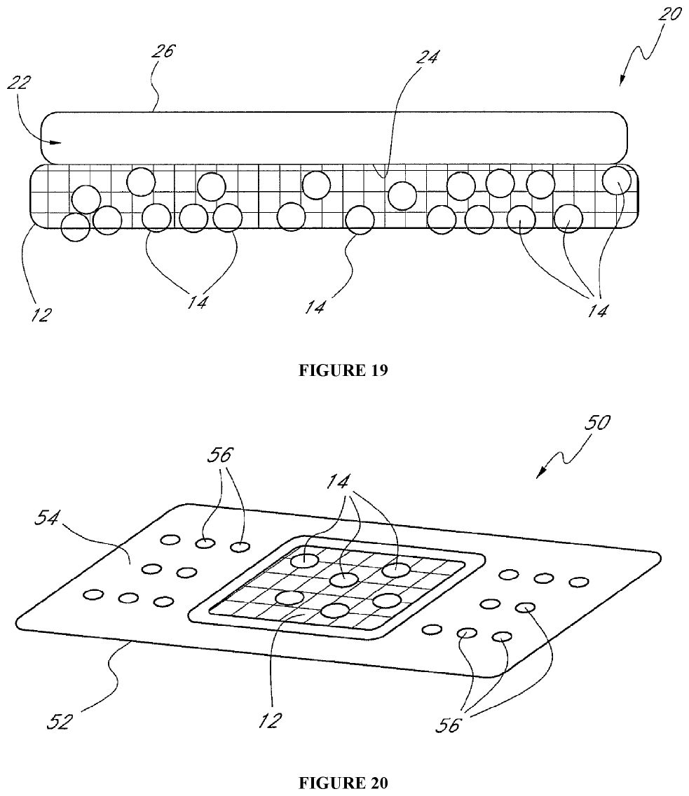

Another embodiment is a pad which is shown at 20 with reference to FIG. 19 and is hereinafter referred to as “pad 20.” The pad 20 comprises the mesh 12, zeolite particles 14 retained therein by the mesh 12, and a support 22 to which pressure may be applied in the application of the pad 20 to a bleeding wound. The mesh 12, as above, has openings that are capable of retaining the zeolite particles 14 therein while allowing the flow of blood therethrough.

The mesh 12 is stitched, glued, clamped, or otherwise mounted to the support 22. The support 22 comprises an undersurface 24 against which the zeolite particles 14 are held by the container 12 and a top surface 26. The undersurface 24 is impermeable to the zeolite particles 14 (migration of the particles into the support 22 is prevented) and is further resistant to the absorption of water or other fluids. The top surface 26 is capable of having a pressure exerted thereon by a person applying the pad 20 to a bleeding wound or by a weight supported on the top surface 26. The entire support 22 is rigid or semi-rigid so as to allow the application of pressure while minimizing discomfort to the patient.

To apply the pad 20 to a bleeding wound, the pad 20 is removed from its packaging and placed on the bleeding wound. As with the pouch of the embodiment of FIGS. 17 and 18, the zeolite particles 14 are either in direct contact with the tissue of the wound or are in direct contact with the blood. Pressure may be applied to the wound by pressing on the top surface 26 with a hand or by placing a weight on the surface, thereby facilitating the contact between the zeolite particles 14 and the wound and promoting the adsorption of the liquid phase of the blood. The pad 20 (with or without a weight) may also be held onto the wound using a strapping device such as a belt, an elastic device, hook-and-loop material, combinations of the foregoing devices and materials, and the like.

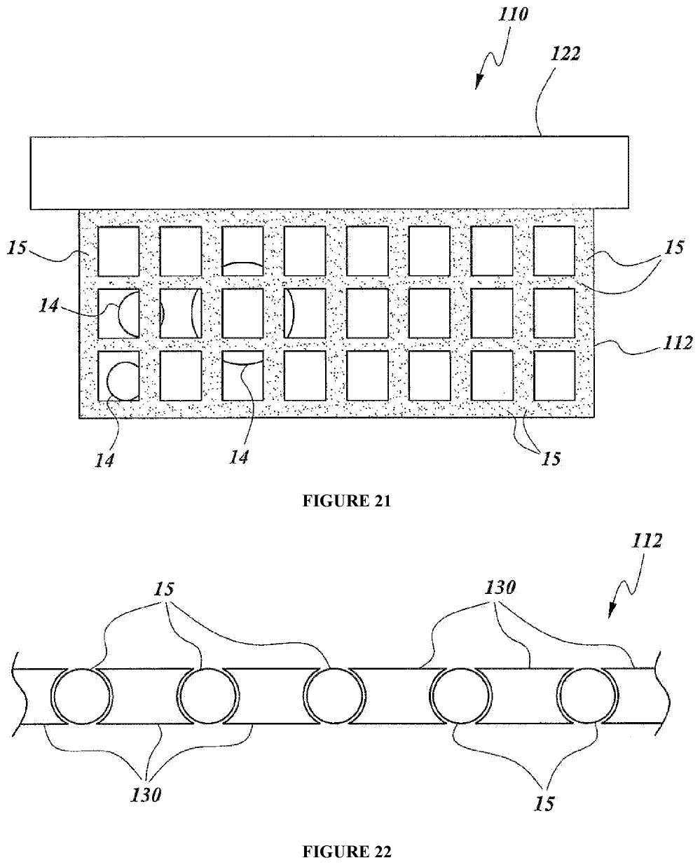

Referring now to FIG. 20, another embodiment is a bandage, shown at 50, which comprises zeolite particles 14 retained in a mesh 12 and mounted to a flexible substrate 52 that can be applied to a wound (for example, using a pressure-sensitive adhesive to adhere the bandage 50 to the skin of a wearer). The mesh 12 is stitched, glued, or otherwise mounted to a substrate 52 to form the bandage 50.

The substrate 52 is a plastic or a cloth member that is conducive to being retained on the skin of an injured person or animal on or proximate a bleeding wound. An adhesive 54 is disposed on a surface of the substrate 52 that engages the skin of the injured person or animal. Particularly if the substrate 52 is a non-breathable plastic material, the substrate may include holes 56 to allow for the dissipation of moisture evaporating from the skin surface.

Referring now to FIG. 21, another embodiment comprises a device 110 having the zeolite particles 14 as described above retained within a fabric pouch. The fabric pouch is a zeolite-impregnated mesh 112 having hemostatic qualities, namely, the hemostatic properties of zeolite. The device 110 may include a support 122, thereby defining a pad. When the device 110 is a pad, the support 122 provides a surface at which pressure may be applied in the application of the device to a bleeding wound. Without the support 122, the device 110 may be used as a surgical sponge.

The zeolite-laden mesh 112 is defined by interconnected strands, filaments, or strips of material that are interconnected by being woven, intertwined, or integrally formed as in the above-disclosed embodiments. The mesh 112 includes particles of zeolite powder 15. Although the particles of zeolite powder 15 are shown as being concentrated along portions of the edges of the mesh 112, it should be understood that the zeolite powder is dispersed throughout the material from which the mesh is fabricated. Preferably, the interconnection of the strands, filaments, or strips to form the mesh 112 is such that the device 110 can flex while substantially maintaining the dimensions of the openings, thereby allowing the zeolite particles 14 to be retained.

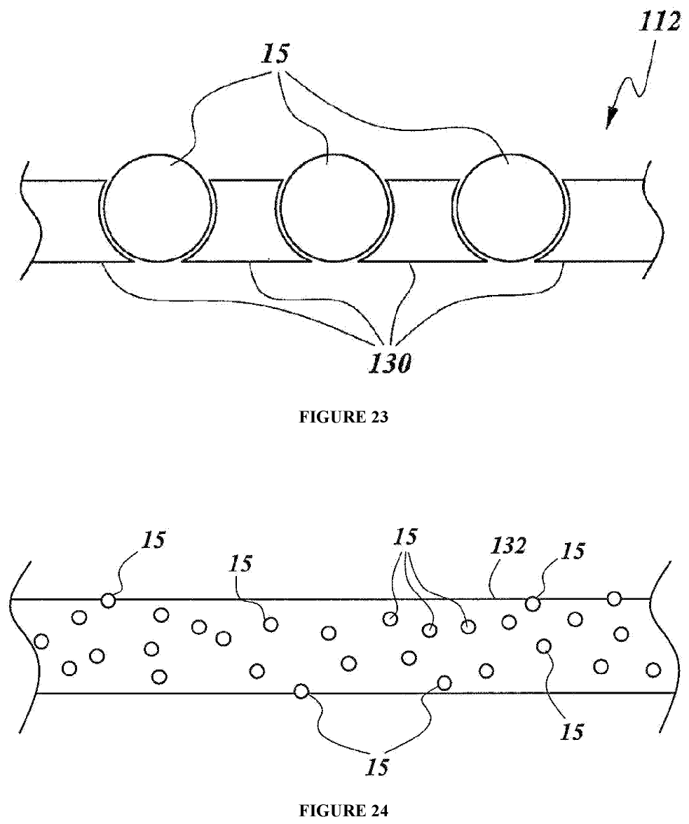

Referring now to FIGS. 22 and 23, zeolite is impregnated into or otherwise retained by the material of the strands, filaments, or strips that define the mesh 112. In particular, the particles of zeolite powder 15 may be captured within a matrix material 130 such that the particles contact the bleeding tissue when the strands, filaments, or strips defining the mesh 112 are brought into contact with the wound. The present invention is not limited to zeolite impregnated or incorporated into the material of the mesh, however, as other materials such as oxidized cellulose may be impregnated or incorporated into the mesh material. As is shown in FIG. 22, the zeolite powder 15 may be captured and held within the outer surface of the matrix material 130. In such an embodiment, the matrix material 130 is preferably sufficiently porous to facilitate the flow of blood therethrough, thus allowing liquid phases of the blood to be at least partially absorbed by the zeolite powder 15 prior to contacting the zeolite particles (or other molecular sieve materials) retained in the mesh 112. As is shown in FIG. 23, the zeolite powder may be captured so as to protrude above the surface of the matrix material 130.

Referring to FIG. 24, the zeolite powder 15 may be impregnated into a substrate material 132 and retained therein by any suitable method. In the impregnation of the zeolite powder 15 into the substrate material 132, the substrate material is generally sufficiently soft (e.g., fluid when exposed to heat) to allow for its deformation to accommodate the zeolite powder. The zeolite powder 15 may be impregnated completely into the substrate material 132, or it may be partially impregnated so as to extend out of the substrate material.

In either the embodiment of FIGS. 22 and 23 or of FIG. 24, the matrix material or the substrate material may be a polymer (e.g., nylon, polyethylene, polypropylene, polyester, or the like), metal, fiberglass, or an organic substance (e.g., cotton, wool, silk, or the like). The matrix material or the substrate material may also be cellulose or a cellulose derivative.

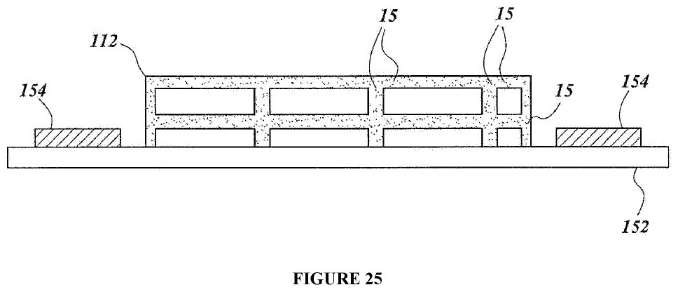

The zeolite-laden mesh 112 may be utilized in conjunction with a bandage, as is shown in FIG. 25. The mesh 112 (which comprises the zeolite powder 15) may be mounted to a flexible substrate 152 that can be applied to a wound in a manner similar to that described above with reference to FIG. 20. The mesh 112 may be stitched, glued, or otherwise mounted to the substrate 152, which may be a plastic or cloth member that is retained on the skin of an injured person or animal on or proximate the bleeding wound (e.g., via an adhesive 154).

In the preparation of zeolite material for the devices of the present invention (i.e., formation of the material into particle form), an initial level of hydration of the zeolite may be controlled by the application of heat to the zeolite material either before or after the material is formed into particles. However, it has also surprisingly been found that as the particle size of the zeolite is increased, the moisture content has less of a correlative effect on any exothermic effect produced as the result of mixing the particlized zeolite in blood. As such, formation of the zeolite material into the zeolite particles (shown at 14 in FIGS. 17-20), may be by extrusion, milling, casting, or the like.

By way of non-limiting illustration, examples of certain embodiments of the present disclosure are given below.

### EXAMPLES

### Example 1: Antimicrobial Activity of Zeolite Nanoparticles (Nanozeolites, NZs)

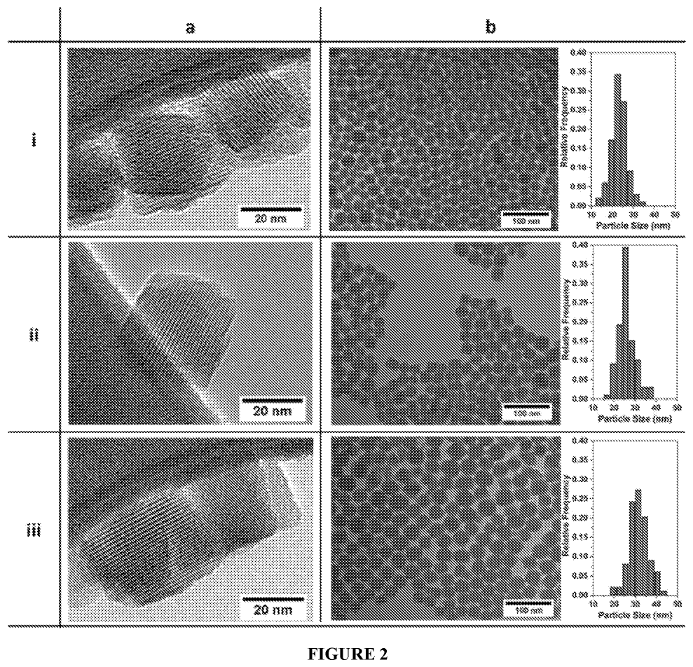

Synthesis of NZs. The synthesis process for the nanozeolites involved a reactor and reactant composition of 0.048 Na₂O: 2.40 (TMA)₂O(2OH): 1.2 (TMA)₂O(2Cl): 4.35 SiO₂: 1.0 Al₂O₃: 249 H₂O (TMA is tetramethylammonium). A process involving 29 h of reflux, water removal and adding the water back resulted in 93% yield of nanozeolite (reported on a synthesis of 10 g scale). FEI Tecnai F20 S/TEM was employed to collect TEM images of nanozeolite samples. ImageJ software was used to measure sizes in TEM images for particle size distribution. FIG. 2 shows the high resolution TEM images produced.

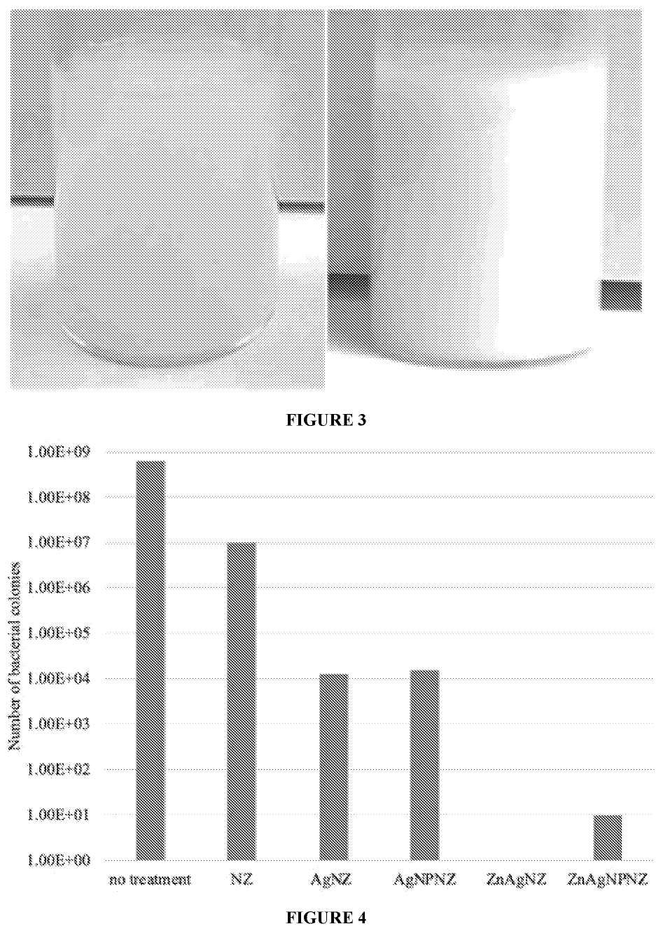

Dispersions of the nanozeolites in water and cyclohexane were prepared, as shown in FIG. 3.

Antimicrobial activity testing. In one example of antimicrobial activity testing, culture was inoculated with 100 μl of bacteria stock and grown overnight in media. The OD at 600 nm of bacteria suspension was measured and translated to number of cells per ml. Bacteria suspension was then added to the media with specified volume ratio. 100 μl of bacterial suspension was added to each test vial containing different amounts of nanozeolite. Bacterial cultures were incubated for 75 minutes at 37° C. with shaking. Bacterial cultures were then removed from incubator and serially diluted in 50% media (50 μl cells/450 μl media) after which 100 μl of given dilutions were plated using glass beads on to agar plates. Plates were incubated overnight at 37° C., and colonies counted (numbers reported on y-axis of plot). The results are shown in FIG. 4.

It is clear from FIG. 4 that in the presence of Zn ions (an adjuvant), the antimicrobial effectiveness is improved. Both Ag ions and AgNPs inhibit growth of E. coli broth cultures at 100 ppm concentrations (ppm concentration here refers to the nanozeolite).

Different Test Surfaces. For antimicrobial activity testing, culture was inoculated with 100 μl of bacteria stock and grown overnight in media. The OD at 600 nm of bacteria suspension was measured and translated to number of cells per ml. Bacteria suspension was then added to the media with specified volume ratio. 200-400 μl of bacterial suspension was added to each test surface. Bacterial cultures were incubated for 75 minutes at 37° C. on the surface. Bacterial cultures were then removed from the surface and serially diluted in 50% media (50 μl cells/450 μl media) after which 100 μl of given dilutions were plated using glass beads on to agar plates. Plates were incubated overnight at 37° C., and colonies counted. FIG. 5 compares the antimicrobial activity on a glass plate, a t-shirt, leather, interior latex paint, and exterior latex paint.

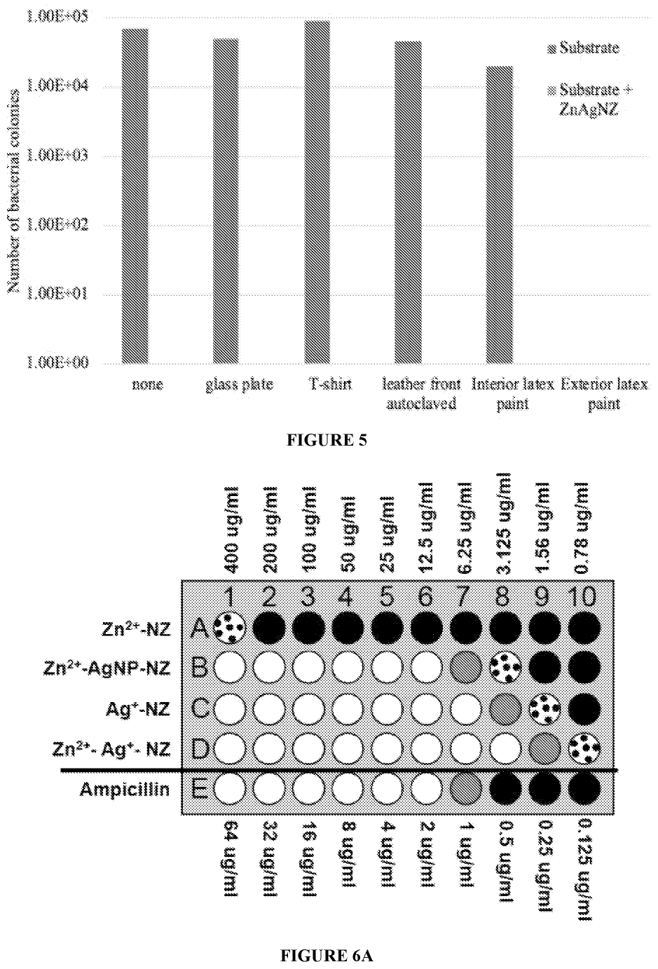

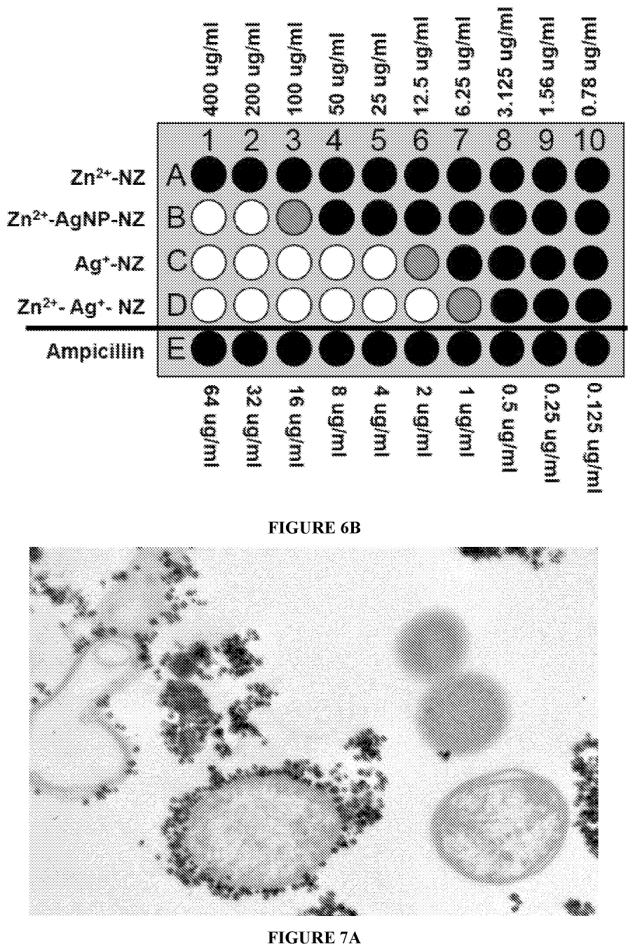

Determination of Minimum Inhibitory Concentrations (MICs). In MIC tests, 50 μl of broth was added to each well in 96-well plate. Nanozeolite samples were sonicated and brought to concentration of 800 μg/ml in LB broth for E Coli and MH2 broth for MRSA. 50 μl of nanozeolites (800 μg/ml) were added to the first well (effectively becoming 400 ppm), mixed 5 times with the pipette, and 50 μl was removed and transferred to the adjacent well. This was repeated for 10-step dilution array. 50 μl of the cell suspension (5×10⁵ cells/ml) was added to each well, halving the concentration of the antimicrobial sample (except the sterility control). Note: the inoculum was determined to be 5×10⁵ cells/ml by OD600. The 96-well round-bottom plate was placed in a 37° C. incubator for overnight incubation. The results for E. coli and MRSA are shown in FIGS. 6A and 6B.

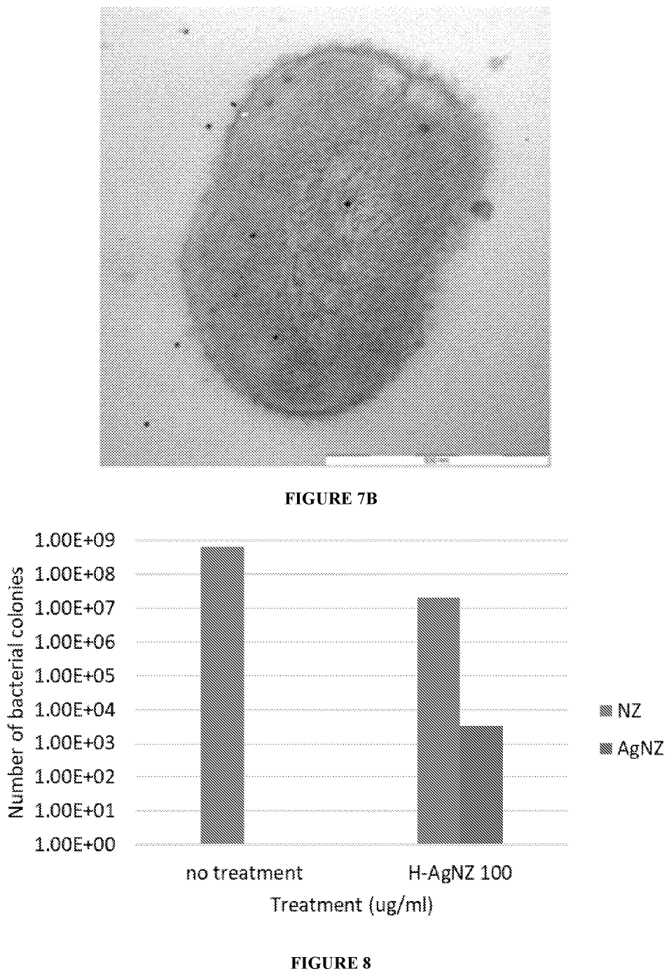

EM with E. coli. Nanozeolites were added at 100 ppm or 500 ppm to E. Coli bacterial cultures and incubated for 15 minutes or 30 minutes before spinning down bacteria and fixing in in 3% glutaraldehyde in 0.2 M sodium cacodylate buffer. The procedure for making the TEM was as follows:
1. Wash twice in cacodylate buffer—10 minutes each
2. Post-fix in 1% Osmium tetroxide in sym-collidine buffer—1 hour
3. Wash twice in sym-collidine buffer—10 minutes each
4. Dehydrate in graded ethanol series—30%, 50%, 70%, 90%, 95%, 3X-100%—10 minutes each
5. Rinse in 100% acetone three times—10 minutes each
6. Infiltrate with 1:2 mixture of acetone: Spurr's epoxy resin overnight
7. Embed in 100% Spurr's resin and polymerize at 80 degrees overnight
8. Section—90 nm thick. Stain sections with Reynolds lead citrate for 3 minutes
9. Examine in JEOL 1400 TEM. Images captured with Olympus Veleta digital camera.
   The resulting micrographs are shown in FIGS. 7A-7B.

Antimicrobial activity testing. In one example of antimicrobial activity testing with proton containing AgNZ, culture was inoculated with 100 μl of bacteria stock and grown overnight in media. The OD at 600 nm of bacteria suspension was measured and translated to number of cells per ml. Bacteria suspension was then added to the media with specified volume ratio. 100 μl of bacterial suspension was added to each test vial containing different amounts of nanozeolite. Bacterial cultures were incubated for 75 minutes at 37° C. with shaking. Bacterial cultures were then removed from incubator and serially diluted in 50% media (50 μl cells/450 μl media) after which 100 μl of given dilutions were plated using glass beads on to agar plates. Plates were incubated overnight at 37° C., and colonies counted (numbers reported on y-axis of plot). The results are shown in FIG. 8.

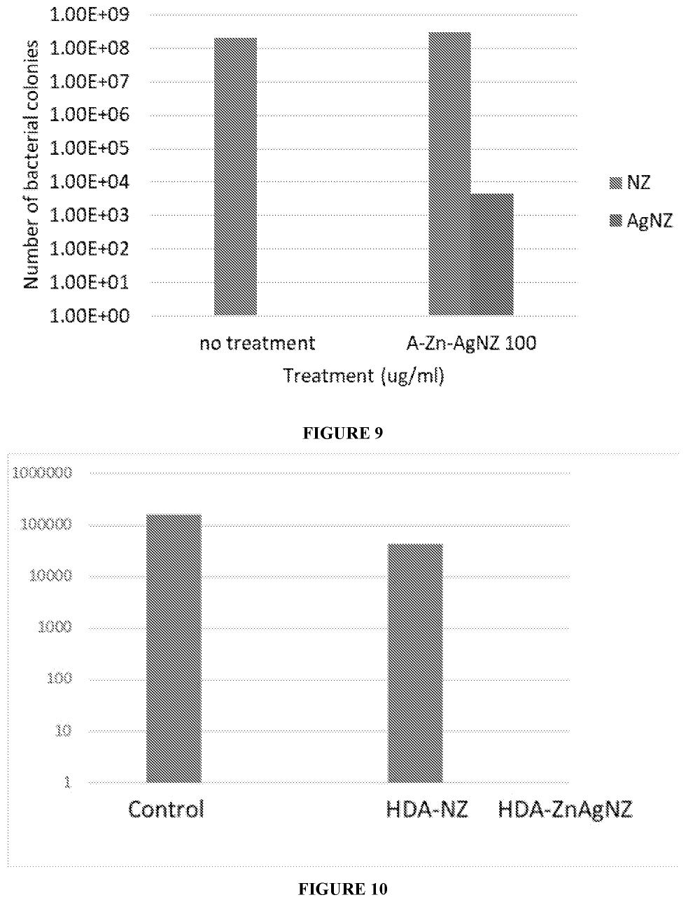

Antimicrobial activity testing. In one example of antimicrobial activity testing with covalent surface functionalized ZnAgNZ (AZNZ), culture was inoculated with 100 μl of bacteria stock and grown overnight in media. The OD at 600 nm of bacteria suspension was measured and translated to number of cells per ml. Bacteria suspension was then added to the media with specified volume ratio. 100 μl of bacterial suspension was added to each test vial containing different amounts of nanozeolite. Bacterial cultures were incubated for 75 minutes at 37° C. with shaking. Bacterial cultures were then removed from incubator and serially diluted in 50% media (50 μl cells/450 μl media) after which 100 μl of given dilutions were plated using glass beads on to agar plates. Plates were incubated overnight at 37° C., and colonies counted (numbers reported on y-axis of plot). The results are shown in FIG. 9.

Antimicrobial activity testing. In one example of antimicrobial activity testing with long-chain hydrocarbon surface modified ZnAgNZ (AZNZ), culture was inoculated with 100 μl of bacteria stock and grown overnight in media. The OD at 600 nm of bacteria suspension was measured and translated to number of cells per ml. Bacteria suspension was then added to the media with specified volume ratio. 100 μl of bacterial suspension was added to each test vial containing different amounts of nanozeolite. Bacterial cultures were incubated for 75 minutes at 37° C. with shaking. Bacterial cultures were then removed from incubator and serially diluted in 50% media (50 μl cells/450 μl media) after which 100 μl of given dilutions were plated using glass beads on to agar plates. Plates were incubated overnight at 37° C., and colonies counted (numbers reported on y-axis of plot). The results are shown in FIG. 10.

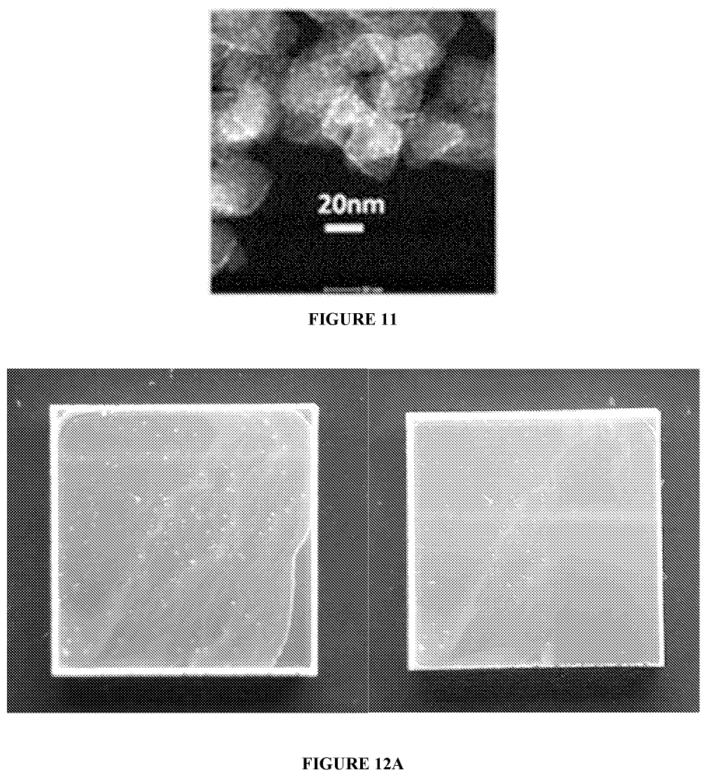

Transmission Electron Microscopy (TEM). FEI Tecnai F20 S/TEM was employed to collect TEM images of silver metal (bright spots) on nanozeolite samples. The results are shown in FIG. 11.

Comparison of Antimicrobial Properties. The antimicrobial properties of the NZs described herein were compared other commercially available antimicrobial silver agents. The results are shown in Table 1 below.

TABLE 1
Comparison of antimicrobial properties.

| MIC of E. coli growth | | | | | Bacterial reduction (E. coli) | |
|---|---|---|---|---|---|---|
| Sample | MIC | | Amount of silver | | Sample | Reduction |
| Zeomic¹ | 62.5 | μg/ml | 1.56 | μg/ml | Silver shield² (amount not specified) | 99% in 2 hours, 25° C. |
| Zn²⁺AgNP-NZ | 3.125-6.25 μg/ml | | 0.26-0.53 | μg/ml | Agion³(amount not specified) | 99.9% (time not specified) |
| | data from duplicate | | | | | |
| Zn²⁺Ag⁺-NZ | 1.56 μg/ml data | | 0.12 | μg/ml | Zn²⁺AgNP-NZ (100 μg/ml) | 99.99999% in 75 min, 37° C. |
| | from duplicate | | | | | |
| 10 nm silver sol⁴ | 10 | μg/ml | 10 | μg/ml | Zn²⁺Ag⁺-NZ (100 μg/ml) | 100% in 75 min, 37° C. |

¹Micron sized Ag⁺ -zeolite A from Zeomic (http://www.zeomic.co.jp/en/product/zeomic)
²Microban product (https://www.microban.com/micro-prevention/technologies/silvershield)
³Micron sized Ag⁺, Zn²⁺ - zeolite A from Agion (http://www.sciessent.com)
⁴American Biotech Labs (https://www.ablsilver.com)
NZ is nanozeolite sample of nanozeolites

From Table 1, it can be clearly seen that the isolated zinc ion silver ion nanozeolite particle (from ZeoVation) is orders of magnitude more effective than micron-sized zeolites and silver colloidal nanoparticles in terms of antimicrobial efficacy. It is also apparent that the zinc ion silver nanoparticle nanozeolite is also very effective

### Example 2: Antimicrobial Activity of Ag⁺, Zn²⁺ Exchanged Zeolite Nanoparticles (AZNZs)

Ag⁺, Zn²⁺ Exchanged Zeolite Nanoparticles (AZNZs) were prepared. These nanoparticles were found to have potent antimicrobial activity.

Efficacy against bacteria in suspension:
3.4×10⁸ CFU/ml E. Coli reduced to 0 CFU/ml in 75 minutes with 100 ppm AZNZ
3×10⁴ CFU/ml E. Coli reduced to 0 CFU/ml with 50, 100, 200 ppm AZNZ in 75 minutes
6×10⁴ CFU/ml MRSA reduced to 50 CFU/ml with 50 ppm AZNZ and 0 CFU/ml with 100, 200 ppm AZNZ in 75 min
100-1000 ppm AZNZ kills 2-3×10⁴ CFU/ml E. Coli in 40 min, does not speed up with concentration
MIC assay: E. Coli 0.8 μg/ml of AZNZ
MIC assay: MRSA 3.1 μg/ml of AZNZ

Efficacy on Surfaces:

An AZNZ suspension was applied to a surface (3.2 μg/cm² of AZNZ), allowed to dry, and exposed to 100,000 E. Coli for 75 min. Liquid was then removed from surface and tested.
Glass—all bacteria killed
Latex painted glass plate—all bacteria killed
T-shirt material—all bacteria killed
Bar towel—no bacteria grew on towel (all liquid absorbed)
Leather—all bacteria killed

Bacteria was also applied to a glass surface, allowed to settle, and then a 3.2 μg/cm² suspension of AZNZ was added. After 75 minutes, all bacteria were killed.

In another example, 6.4 and 3.2 μg/cm² AZNZ suspensions were applied to glass surfaces (smooth and textured), allowed to dry, wiped gently with dry and wet cloth. Bacteria was then applied to the surface; however, no bacterial growth on the surface was observed.

These compositions could be used to provided surfaces resistant to bacterial growth, including MRSA growth. In another experiment, 32 μg/cm² of AZNZ was applied to various surfaces. The surfaces were then exposed to 400,000 bacteria (MRSA). In the case, of a glass surface, MRSA was reduced by a factor of 40 (9×10³ CFU/ml). In the case of a bar rag, MRSA was reduced to 30 CFU/ml.

Based on these results, the following compositions have been proposed and evaluated:

Sprays (made with neutral detergent) containing AZNZ were transparent, odorless, and colorless, and could sterilize surfaces and provide continuing antimicrobial effects following drying of the spray.

Paints—water-based paint with surface modified (hydrophobically modified) AZNZs were found to work well. It is believed that surface derivatization moves AZNZ to surface as the paint dries on wood, drywall, resulting in a coating that is resistant to bacterial growth (including E. Coli and MRSA growth).

Oil-Based Paints—when applied to metal (introduced as spray in toluene), the system worked with external lab testing.

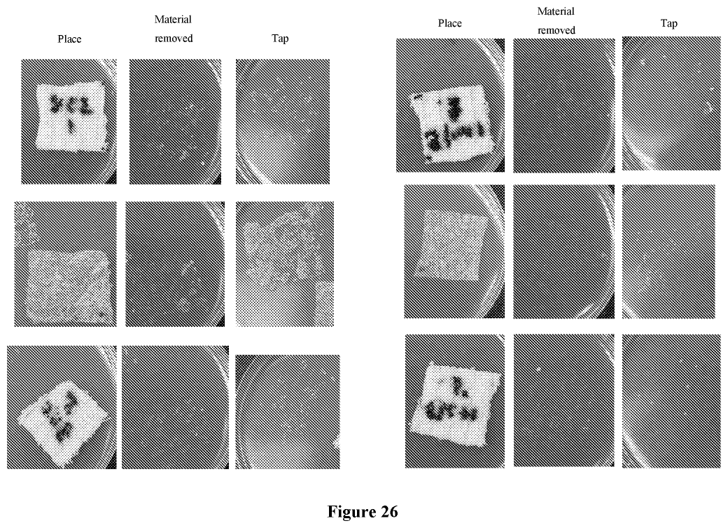

Heat-activated transfer sheets (FIG. 26)—transfer of antibacterial activity from sheet to cloth successful. Quantitate decrease of MRSA by a factor of 10, also qualitative experiments showed decrease in colonies. The surface derivatized AZNZ seemed to perform better (qualitative assessment). Amount of silver (not AZNZ) on dryer sheet was 0.1 μg/cm² (applied 2.3 μg/cm², not being retained), whereas for glass and bar towel, it was 6 μg/cm² of silver. Need better technology to transfer and retain AZNZ on dryer sheet

Bandages: Capable of killing E. Coli and MRSA

Gel-based wound dressing: Capable of killing E. Coli.

### Example 3: Antimicrobial Compositions

Described herein is a water-based spray that renders a surface antimicrobial, is resistant to abrasion, and with a built-in indicator to indicate when the antimicrobial nature of the surface is diminished. The example spray includes:
1) Water as the base carrier;
2) A nonionic surfactant that helps the spray disperse uniformly on the surface;
3) Silver and zinc ion-exchanged nanozeolites dispersed as a stable colloid in the water (which functions as the active antimicrobial agent);
4) A binder polymer at a low concentration that helps the adhesion of the nanozeolite to the surface; and
5) An optical tracer (organic dye) molecule associated with the nanozeolite surface that fluoresces in the visible upon illumination by a UV lamp. The fluorescent color is visible to the naked eye and can also be detected by a suitable photometric detector.

Method of application: The spray can be applied to a hard or soft surface. The amount applied is dependent on the environment and can very from 2-20 sprays over a given area. Hard surfaces include glass, wood, plastic, drywall, floors. Soft surfaces include textiles, leather, tent material.

Mode of antimicrobial activity: The antimicrobial activity arises from the silver-zinc nanozeolite particles stuck on the surface that interacts with the pathogens and kills by contact.

Increasing stickiness to the surface: The spray formulation contains a small concertation of binder polymer that aids in the stickiness of the nanozeolite to the surface. The concentration of the binder polymer should be sufficiently low not to form a thick film on the surface, and cover up the zeolite particles.

Optical Tracer (fluorescent sensor): A tracer (e.g., a dye molecule) is associated with the zeolite nanoparticles via adsorption, electrostatic or covalent bonding. Any loss of the zeolite from the surface will lead to the loss of fluorescence and is therefore a measure of the amount of zeolite resident on the surface.

Preliminary Data

Preliminary experiments on the stickiness and detection of the spray were carried out on glass, metal and wood plates. The data is included below.

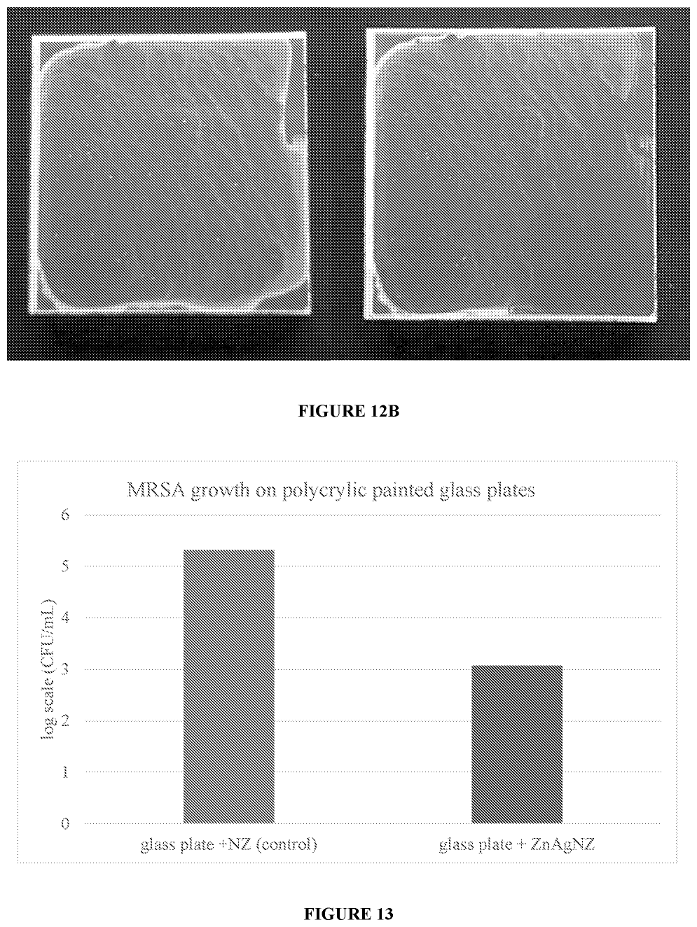

On the plate, the spray material was applied, and allowed to dry. In each case, a dry cloth is put under a 100 g weight and half of the plate is rubbed (no vertical force is applied). The data are shown in FIGS. 12A-12B for glass. Each figure has two parts, the left-hand part is the fluorescence image (with a hand-held UV lamp), and the right-hand part is after one-half of the plate is rubbed for 20 rubs. FIG. 12A is the antimicrobial zeolite along with the dye, FIG. 12B has the antimicrobial zeolite along with the dye and the binder. The blue fluorescence is an indication of the presence of the antimicrobial nanozeolite on the glass surface. In the case of zeolite and dye alone, the abrasion force was enough to remove the fluorescence and thereby the nanozeolite. In the case of the sample with the binder, the fluorescence of the left half of the plate is better retained, indicating that the zeolite is held more strongly to the surface in the presence of the binder. Similar data was obtained for wood and metal, respectively.

From these experiments, it is clear that there is increased adhesion of the nanozeolite in the presence of the binder, and that the dye is a good indicator of the amount of antimicrobial nanozeolite on the surface.

Antimicrobial data: The glass samples were further tested for antimicrobial activity towards MRSA. MRSA was chosen because it is most difficult to kill and has a thick peptidoglycan layer. FIG. 13 plots the data for 200 μl/cm² of 3000 ppm antimicrobial zeolite along with the binder and the dye that was dried on the glass surface, prior to the pathogen testing. 300 μl of MRSA containing suspension was then added to the glass plate (1.5×10⁵ cells), the sample was left for 75 minutes, and then the glass plate was introduced into a thioglycolate containing broth. After subsequent dilutions, and plating on to agar plates, the colonies were counted after 24 hours. We are getting greater than a 2-log reduction in 75 minutes. We expect better results with higher concentrations and longer times.

### Example 4. Antiviral Zeolite Nanoparticles and Viricidal Coatings Formed from Zeolite Nanoparticles

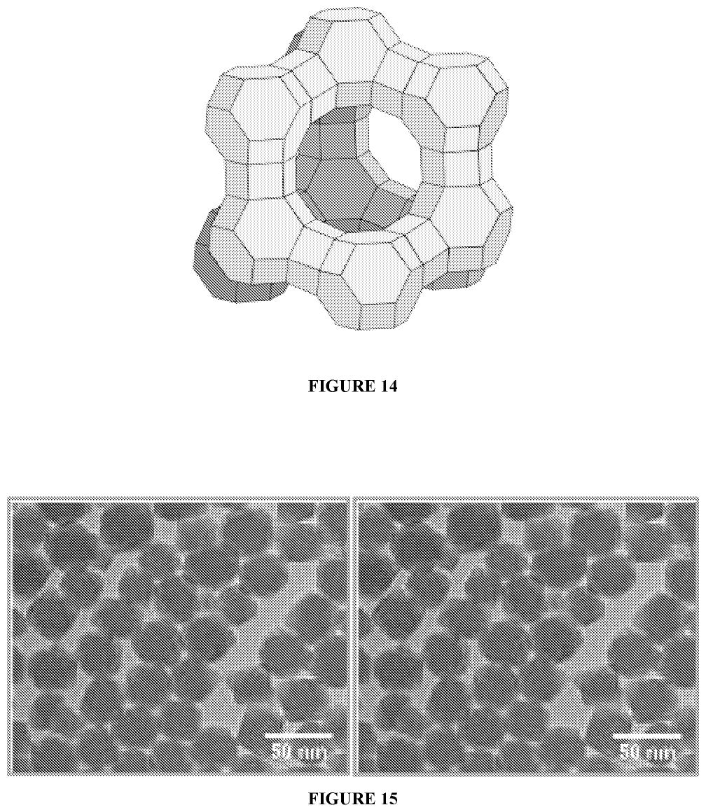

The coronavirus disease 2019 (COVID-19) was first described in late December 2019 in Wuhan, China. There is a need to create antimicrobial/antiviral coatings for hard and textiles surfaces that are high contact, high traffic areas. In this example, coatings formed from zeolite nanoparticles are described. The coatings will exhibit antiviral activity. Further, the proposed surface coating technology will no longer necessitate wiping surfaces to render them clean Transition metal ions, including Ag⁺, Zn²⁺, Cu²⁺, Fe^{2+/3+}, as well as oxides of Al³⁺, Ti⁴⁺, Ga³⁺ have demonstrated antimicrobial, antiviral, antifungal and antiparasitic activity. Combination of these ions will often show synergy effects, significantly improving activity. However, delivery of these ions to the relevant sites can be problematic, since they are reactive in their isolated forms and can be neutralized by the environment, e.g. by precipitation. Zeolites are highly porous crystalline aluminosilicates of composition M_{n/m}^{m+}. Si₁₋ₙAlₙO₂.xH₂O, with the framework made up of interconnected TO₄ (T=Si, Al) tetrahedra. Introduction of Al³⁺ into the structure results in a negatively charged framework, with extra-framework ion-exchangeable M^{m+} providing charge neutrality (FIG. 14). By ion-exchange process, the cations in the as-synthesized zeolite can be replaced with transition metal cations, including Ag⁺, Zn²⁺, Cu²⁺, Fe^{2+/3+}, Ga³⁺. These ions are polarized by the strong electric fields within the framework, and results in strong attraction between the transition metal ions and the zeolite framework Nanozeolites, such as those described herein, can serve as a reservoir of these ions, directly delivering them to a pathogenic agent. FIG. 15 shows a high resolution TEM of nanozeolites. The zeolite size facilitates formation of stable, aqueous dispersions for spray applications while protecting the metals. These nanozeolites can be prepared using a high efficiency synthesis with much improved yields and times of synthesis.

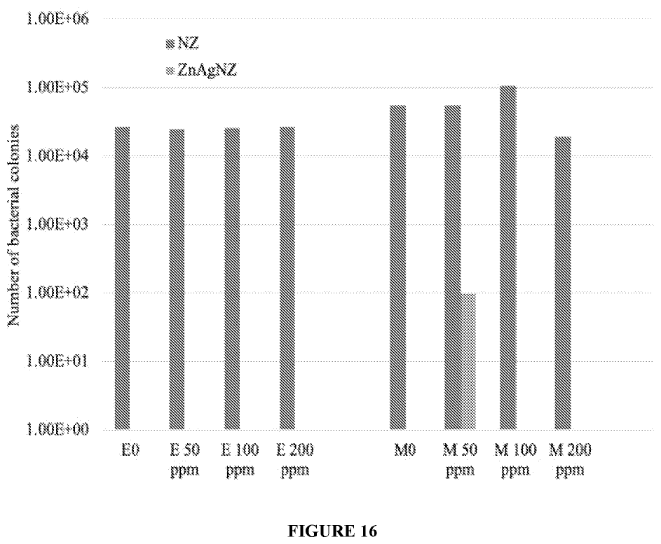

Ion-exchanged Ag⁺/Zn²⁺ nanozeolite (ZnAgNZ) has been evaluated for antimicrobial and antifungal spray applications, and demonstrated at least 100-fold higher antimicrobial potency over current zeolite-based antimicrobials and a factor of 1000 over silver colloids (per unit weight of silver). The minimum inhibitory concentration towards E. coli and methicillin resistant S. aureus (MRSA) were 780 μg/ml and 6.25 μg/ml of ZnAgNZ. These are some of the lowest values of MIC reported in the literature. In addition, excellent activity towards Candida albicans, as well as a mixture of fungi was noted. The high activity stems from three factors: strong binding of the high-surface-area zeolite with the cell membrane, penetration of the nanozeolite directly into the bacteria, and rapid ion-exchange kinetics of the transition metal ion load out from the zeolite (due to the shorter diffusion length) to attack the pathogen. Example of the activity towards E. coli and MRSA is shown in FIG. 16. Silver ions, in particular are a broad-spectrum antimicrobial, effective against gram positive, gram negative bacteria (including methicillin- and vancomycin resistant bacteria) as well as fungi, viruses, and biofilms, often at micromolar concentrations. In general, Gram-positive bacteria (e.g. S. aureus) is more resistant than Gram-negative bacteria (E. coli), due to the thicker peptidoglycan layer.

The mechanism of activity of silver ions is complex. Since there are many points of attack on the cell, with chemistry within the cell as well as cell surface, this multi-pronged effect of Ag⁺ is responsible for antimicrobial activity. This is because Ag⁺ has a strong propensity to form complexes with ligands containing S, N and O. Thus, biologically relevant species, such as thiols, carboxylic acids, phosphates, amines will act as ligands for silver ion. In addition, Ag⁺ can also compete with the native binding metals in enzymes, particularly the iron-sulfur clusters of bacterial enzymes involved in amino acid synthesis and with DNA bases. It inhibits both phosphate uptake and exchange, as well as causing the efflux of succinate, glutamine, and proline in E. coli. Silver ions also disrupt the proton gradients across membranes that are necessary for metabolic activity. Collapse of the proton gradient increases cell respiration, and becomes uncouples from ATP-dependent processes. Ag⁺ also inhibits energy-dependent Na⁺ transport by binding with the Na⁺-translocating NADH:ubiquinone oxidoreductase, as demonstrated in Bacillus sp. strain FTU and Vibro Alginolyticus. Ag⁺ is therefore bactericidal at very low concentrations and not necessarily arising from binding to a specific target, but non-specific binding to membrane proteins and/or the phospholipid bilayer.

Another mechanism proposed for antimicrobial activity of silver involves the formation of reactive oxygen species. Upon Ag⁺ exposure, electron microscopy studies of Gram-negative Escherichia coli (E. coli, ATCC 23282) and Gram-positive Staphylococcus aureus (S. aureus, ATCC 35696) shows that the DNA appears to be aggregated in the center of the cell. Either localized or complete separation of the cell membrane from the cell wall upon treatment with Ag⁺ has been noted.

In this example, the antiviral activity of the transition metal-zeolite sprays will also be evaluated. These zeolite formulations will exhibit activity towards SARS-CoV-2 (the pathogen causing COVID-19). Silver/copper ion zeolites (micron sizes) cause significant reductions of human coronavirus 229E, yielding greater than 5-log reduction within 24 h. The mechanism of action was not studied, but it was speculated that the silver ions may bind to the sulfhydryl groups, modify the adsorption of viruses to host cells, the host cell receptors may be blocked, or the nucleic acid within the capsid may be inactivated. Copper can block functional groups on the proteins and inactivate enzymes, and also create ROS, based on Fenton chemistry. Studies have shown that silver zeolites can (375 mg/1) effectively neutralize SARS-CoV-P11 and SARS-CoV-P8 strains of virus within two hours. Since viruses do no possess resistance or repair mechanisms, they are very susceptible to effects of transition metal ions.

Additionally, via the Fenton reaction, and inclusion of hydrogen peroxide, metal impregnated zeolites can produce free radicals from hydrogen peroxide, causing pathogen death, providing extended antipathogen protection.

Objectives and Aims

Goal: To demonstrate the antimicrobial and antiviral activity of an aqueous zeolite spray.

Aims:
1) Synthesis of zeolites, transition-metal exchanged zeolites, characterization and formulation of spray solutions.
2) Assess the acute antimicrobial efficacy of zeolites on textiles such as cotton and canvas, and hard surfaces such as wood, metal, plastic, and other synthetics in a controlled laboratory setting.
3) Determine if combination of peroxide and transition metal zeolites can have antipathogenic activity

Aim 1

Zeolite synthesis will be carried out with the following composition 0.048 Na₂O: 2.40 (TMA)₂O(2OH): 1.2 (TMA)₂O(2Cl): 4.35 SiO₂: 1.0 Al₂O₃: 249 H₂O (TMA-tetramethylammonium ion). Following zeolite synthesis, ion-exchange with transition metal ions will be carried out. Silver ions will be the common ion, because of its potent activity and the co-cations will be studied to examine synergistic enhanced activity. The following materials will be prepared by ion-exchange (saturation ion-exchange with Ag⁺, followed by ion-exchange of the second cation)
1) Ag⁺/Zn²⁺-zeolite
2) Ag⁺/Cu²⁺-zeolite
3) Ag⁺/Fe²⁺-zeolite
4) Ag⁺/Fe³⁺-zeolite
5) Cu²⁺/Fe²⁺-zeolite (this material is relevant because of the potential to do Fenton Chemistry and release toxic hydroxyl radicals)

All materials will be characterized by X-ray diffraction (to ensure zeolite structure), transmission electron microscopy (to ensure zeolite size), and elemental analysis by atomic absorption spectroscopy (to calculate loading levels of transition metal). Aqueous sprays with 1000-3000 ppm zeolite with different transition metal ions will be studied.

Aim 2

Using an already established model, zeolites will be sprayed onto different textile and hard surfaces and quantified for bacterial growth against both Gram + (Staphylococcus aureus) and Gram − (Escherichia coli). Briefly, zeolites will be sprayed onto different textile and hard surfaces and quantified for inhibition of growth against both Gram + (e.g. Staphylococcus aureus) and Gram − (e.g. Escherichia coli) bacteria (12-60 pg of zeolite/cm² is the range of loading levels on the surface). Treated textiles and surfaces will be inoculated with bacterial cultures of known titer by spreading equal aliquots of culture across the surface of test samples of equal size. The soiled samples will then be held at 4° C., room temp (approximately 22° C.) and 37° C. for times ranging from 2 hr to 24 hr. After the holding period, samples will be suspended in a volume of sterile saline enough to cover the sample completely. The samples will be vortexed to recover any bacteria. The supernatants will be titered by serial dilution on TSA plates, and CFU/sample will be calculated to determine the number of live bacteria remaining. Alternatively, the samples could be briefly (e.g. 10 min) immersed in a bacterial culture, then removed and held for various times, and analyzed as described. Additionally, treated textiles and surfaces could be tested for anti-fungal activity using Candida albicans and Aspergillus niger by similar methods.

In addition, studies will be performed to determine reduction of SARS-CoV-2 viral load prior and after decontamination of materials and surfaces using TCID and plaque assay.

Aim 3

To assess if formation of reactive oxygen species hypothesized to form due to Fenton reaction of metals contained in the zeolites under oxidative environments, the efficacy of optimized zeolite spray coatings will be improved.

### Example 5: Use of Zeolite Nanoparticles in to Treat Hemorrhagic Events

Hemorrhagic events can occur during medical procedures, accidents, conflicts and wars and can be minor or life threatening. Some of the technologies that are available include:

(1) use of bandages with chitosan (aminopolysaccahride fiber obtained from shellfish), which has coagulant properties due to its cationic nature and mucoadhesive properties;

(2) bandage dressings made from fibrin, thrombin, and factor XIII purified from human donated blood and plasma promotes natural clot formation and is quite effective in models of severe arterial bleeding;

(3) bandages that incorporate a derivative from sea algae to promote hemostasis;

(4) flexible dressings that include a mixture of mineral components, including bentonite, kaolinite and illite or attapulgite, and may include anti-fungal (or other) agents; granular forms of zeolite poured into a wound absorbs water from hemorrhaged blood and concentrates hemostatic factors in the blood at the site of injury. In addition, the heat from the exothermic reaction promotes stoppage of blood flow by cauterization; and

(5) powder consisting of microporous beads which absorb water and concentrate clotting factors.

Described herein are compositions comprising of a fractal network of zeolite nanoparticles (e.g., 30 nm nanoparticles) which, when applied to a bleeding area will absorb liquid and promote blood clotting. The fractal network of nanozeolites can provide better heat dissipation through the wound, and minimize burns. The fractal network includes, for example, 30 nm zeolites, which together form micron sized particles. Particles with the fractal nanozeolite network can have an average diameter of from about 0.2 mm to about 10 mm, with moisture content varying from 1 to 20%. The nanozeolites can be ion-exchanged with silver, zinc, copper, calcium and other ions, as needed. The ion concentration can vary from 1 to 20 wt %. In addition, other materials including antibiotics, antifungal agents, antimicrobial agents, anti-inflammatory agents and analgesics can be included.

The nanozeolites can be applied to wounds via a variety of suitable methods. Examples of suitable methods include:

(1) The fractal network of nanozeolites can in the form of granules, powder, micron beads, liquid, paste, gel, impregnated in a bandage, and electrospun into a bandage. The composition may further comprise one or more substances such as, for example, clay binders, superabsorbent polymers, chitosan, fibrinogen, thrombin, calcium, vasoactive catecholamines, vasoactive peptides, electrostatic agents, antimicrobial agents, anesthetic agents, fluorescent agents, and quick dissolve carrier polymers such as dextran and polyethylene glycol (PEG);

(2) Methods of making a bandage scaffold: 1) a hydrogel (e.g., gelatin) as the carrier structural polymer that will dissolve rapidly in the wound (200 mg/1 ml solvent) 2) nanozeolite (300 mg) 3) super-adsorbent polymers, blends of crosslinked polyacrylic acid/salts, which form a gel that dissolves quickly in water (1 mg) 4) glutaraldehyde vapor to fix the scaffold;

(3) Powder in sterilized packaging deposited directly at the points from which blood emanates to dress the wound;

(4) Examples of apparatus containing the nanozeolites include sleeves for arm wounds, stocking for foot wounds, caps for head wounds with the nanozeolite networks contained in a mesh with binder materials such as crosslinked calcium alginate and positioned so that the zeolite contacts the wound; and

(5) Nanozeolites dispersed in an aqueous medium (5-10 wt %) that can be directly applied to the wound and also as a spray form and also as foam form to promote clotting.

### Example 6: Use of Zeolite Nanoparticles in Wound Healing

Nanozeolites can be used to suppress inflammation, control microbial infections, and accelerating wound healing. Nanobridging by nanozeolite particles can bring rapid and strong closure and healing of wounds.

This can be accomplished by controlled release of silver and other ions, e.g. calcium from the nanozeolite promoting control collagen deposition leading to a proper alignment of collagen fibrils that accelerates wound healing. Slow release of ions from the nanozeolite minimizes toxic range for keratinocytes and fibroblast. In addition, nanozeolites can act as a bridge between the wound surfaces.

The nanozeolites can be applied to wounds via a variety of suitable methods. Examples of suitable methods include:

(1) Nanofibers containing nanozeolites with a high surface area and porosity, enhance the wound exudate absorption capability, breathability of the dressing. Nanofeature mimics the topography of the endogenous extracellular matrix (ECM), therefore supporting fibroblasts and keratinocytes attachment and proliferation facilitating collagen synthesis and ultimately re-epithelization. Nanofibers to be made by electrospinning;

(2) Hydrogels (polysaccharides) superabsorbent dressings containing nanozeolites enhance autolytic debridement because they are mucoadhesive and naturally possess high water affinity;

(3) Nanocomposites made from polymers, nanozeolites and water (e.g. nanozeolite-polyvinylpyrrolidone gel) cross-linking the gel or a freeze-dried dressing material, applied to the wound, whereas the polymer releases the nanozeolite into the wound;

(4) Applied as an aqueous suspension of nanozeolite to the wound. The nanozeolite particles adsorbed onto the wound surface because of its affinity with the wound surface. Strong irreversible anchoring of the nanozeolites to the wound surface will be possible by controlling the surface chemistry of the nanozeolite. The edges of the wound need to be brought together manually and kept in contact.

### EXAMPLE EMBODIMENTS

1. An antimicrobial composition comprising water and a population of modified zeolite nanoparticles dispersed therein, wherein the zeolite nanoparticles comprise an effective amount of antimicrobial metal ions to kill or inhibit the growth of a microbe.
2. The composition of embodiment 1, wherein the modified zeolite nanoparticles comprise a surface that has been modified via association of a hydrophobic capping molecule.
3. The composition of embodiment 2, wherein the capping molecule comprises a hydrophobic molecule comprising a cationic moiety, and wherein the cationic moiety is electrostatically associated with the surface of the zeolite.
4. The composition of embodiment 3, wherein the capping molecule comprises an amine defined by Formula I or Formula II below

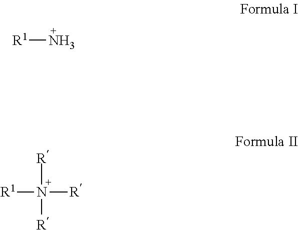

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

5. The composition of embodiment 2, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.
6. The composition of embodiment 2, wherein the surface is covalently modified via reaction with a halosilane selected from octadecyltrichlorosilane (OTS), hexyltrichlorosilane (HTS), ethyltrichlorosilane (ETS), and combinations thereof.
7. The composition of any of embodiments 1-6, wherein the zeolite nanoparticles have an average diameter of less than 100 nm, such as from 10 nm to less than 100 nm, or from 20 nm to 60 nm.
8. The composition of any one of embodiments 1-7, wherein the antimicrobial metal ions comprise metal nanoparticles formed from an antimicrobial metal.
9. The composition of embodiment 8, wherein the antimicrobial metal comprises silver, copper, zinc, or a combination thereof.
10. The composition of any of embodiments 8-9, wherein the metal nanoparticles have an average diameter of 10 nm or less, such as from 1 nm to 10 nm, or from 1 nm to 5 nm.
11. The composition of any of embodiments 8-10, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
12. The composition of any one of embodiments 1-11, wherein the antimicrobial metal ions comprise antimicrobial metal ions retained at ion-exchangeable sites within the zeolite nanoparticles.
13. The composition of embodiment 12, wherein the antimicrobial metal ions include copper ions, zinc ions, silver ions, or a combination thereof.
14. The composition of any one of embodiments 12-13, wherein the antimicrobial metal ions are present in an amount of 10% or greater of the ion exchange capacity of the zeolite nanoparticles, such as from 50% up to 100% of the ion exchange capacity of the zeolite nanoparticles.
15. The composition of any one of embodiments 1-14, wherein the zeolite nanoparticles have an average internal surface area of at least 300 m²/g.
16. The composition of any one of embodiments 1-15, wherein the zeolite nanoparticles further comprise an adjuvant.
17. The composition of embodiment 16, wherein the adjuvant includes a small molecule antimicrobial agent.
18. The composition of any one of embodiments 1-17, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
19. The composition of any one of embodiments 1-18, wherein the composition further comprises a non-ionic or zwitterionic surfactant.
20. The composition of embodiment 19, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.
21. The composition of any one of embodiments 1-20, wherein the composition further comprises a plurality of polymer particles dispersed in the water.
22. The composition of embodiment 21, wherein the plurality of polymer particles comprise a copolymer having a theoretical T_{g} of from −10° C. to 50° C., such as from 10° C. to 45° C., or from 17° C. to 35° C.
23. The composition of embodiment 22, wherein the copolymer is derived from one or more ethylenically-unsaturated monomers selected from the group consisting of styrene, butadiene, meth(acrylate) monomers, vinyl acetate, vinyl ester monomers and combinations thereof.
24. The composition of any one of embodiments 22-23, wherein the copolymer is an acrylic-based copolymer.
25. The composition of any one of embodiments 22-24, wherein the copolymer is derived from:

(i) one or more (meth)acrylate monomers;

(ii) one or more carboxylic acid-containing monomers;

(iii) optionally one or more additional ethylenically-unsaturated monomers, excluding monomers (i) and (ii).

26. The composition of embodiment 25, wherein the copolymer is derived from greater than 80% by weight of one or more (meth)acrylate monomers, based on the total weight of all of the monomers used to form the copolymer.
27. The composition of any one of embodiments 25-26, wherein the one or more (meth)acrylate monomers are selected from the group consisting of methyl methacrylate, butyl acrylate, 2-ethylhexylacrylate, and combinations thereof.
28. The composition of any one of embodiments 25-27, wherein the one or more carboxylic acid-containing monomers are selected from the group consisting of acrylic acid, methacrylic acid, itaconic acid, maleic acid, fumaric acid, and combinations thereof.
29. The composition of any one of embodiments 1-28, wherein the composition further comprises a pigment, a filler, a coalescent, a co-solvent, a volatile base, or a combination thereof.
30. The composition of any one of embodiments 1-29, wherein the composition comprises a paint, such as a semi-gloss paint.
31. The composition of any one of embodiments 1-20, wherein the composition comprises an antimicrobial spray.
32. An antimicrobial composition comprising a hydrophobic carrier and a population of zeolite nanoparticles dispersed therein, wherein the zeolite nanoparticles comprise an effective amount of antimicrobial metal ions to kill or inhibit the growth of a microbe.
33. The composition of embodiment 32, wherein the zeolite nanoparticles are hydrophilically modified.
34. The composition of any one of embodiments 32-33, wherein the hydrophilically modified zeolite nanoparticles comprise a surface that has been modified via association of a hydrophilic capping molecule.
35. The composition of embodiment 34, wherein the capping molecule comprises a hydrophilic molecule comprising a cationic moiety, and wherein the cationic moiety is electrostatically associated with the surface of the zeolite.
36. The composition of embodiment 34, wherein the surface is covalently modified via reaction with a hydrophilic alkoxysilane, a hydrophilic halosilane, or a combination thereof.
37. The composition of any of embodiments 32-36, wherein the zeolite nanoparticles have an average diameter of less than 100 nm, such as from 10 nm to less than 100 nm, or from 20 nm to 60 nm.
38. The composition of any one of embodiments 32-37, wherein the antimicrobial metal ions comprise metal nanoparticles formed from an antimicrobial metal.
39. The composition of embodiment 38, wherein the antimicrobial metal comprises silver, copper, zinc, or a combination thereof.
40. The composition of any of embodiments 38-39, wherein the metal nanoparticles have an average diameter of 10 nm or less, such as from 1 nm to 10 nm, or from 1 nm to 5 nm.
41. The composition of any of embodiments 38-40, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
42. The composition of any one of embodiments 32-41, wherein the antimicrobial metal ions comprise antimicrobial metal ions retained at ion-exchangeable sites within the zeolite nanoparticles.
43. The composition of embodiment 42, wherein the antimicrobial metal ions include copper ions, zinc ions, silver ions, or a combination thereof.
44. The composition of any one of embodiments 42-43, wherein the antimicrobial metal ions are present in an amount of 10% or greater of the ion exchange capacity of the zeolite nanoparticles, such as from 50% up to 100% of the ion exchange capacity of the zeolite nanoparticles.
45. The composition of any one of embodiments 32-44, wherein the zeolite nanoparticles have an average internal surface area of at least 300 m²/g.
46. The composition of any one of embodiments 32-45, wherein the zeolite nanoparticles further comprise an adjuvant.
47. The composition of embodiment 46, wherein the adjuvant includes a small molecule antimicrobial agent.
48. The composition of any one of embodiments 32-47, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
49. The composition of any one of embodiments 32-48, wherein the composition further comprises a non-ionic surfactant.
50. The composition of embodiment 49, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.
51. The composition of any one of embodiments 32-50, wherein the hydrophobic carrier comprises a siccative oil, an alkyd resin, or a combination thereof.
52. The composition of embodiment 51, wherein the siccative oil has an iodine number of at least 115 (e.g., an iodine number of from 115 to 180), such as an iodine number of at least 130 (e.g., an iodine number of from 130 to 180).
53. The composition of embodiment 52, wherein the siccative oil comprises linseed oil, tung oil, or a combination thereof.
54. The composition of any one of embodiments 32-53, wherein the composition further comprises a pigment, a filler, a co-solvent, or a combination thereof.
55. The composition of any one of embodiments 32-54, wherein the composition comprises a paint, such as a semi-gloss paint.
56. A method of producing a coating on a surface comprising:

(a) applying to the surface the composition of any of embodiments 1-55; and

(b) allowing the composition to dry to produce the coating.

57. The method of embodiment 56, wherein the coating kills or inhibits the growth of a microbe.
58. The method of any one of embodiments 56-57, wherein the microbe is selected from a bacteria, a fungi, a virus, an algae, or a combination thereof.
59. The method of embodiment 58, wherein the microbe is a bacteria selected from Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas fourescens, Nocardia brasiliensis, Nocardia globerula, Acinetobacter genomospecies, Acinetobacter calcoaceticus, Acinetobacter baumannii, Stenotrophomonas maltophlia, Pantoea stewarti ss stewartii, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof.
60. The method of embodiment 58, wherein the microbe is a fungi selected from Candida albicans, Candida parapsilosis, Candida tropicalis, Candida glabrata, Kluyveromyces marxianus, Hyphopichia burtanii, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof.
61. A dryer sheet comprising

a nonwoven substrate; and

a transferrable carrier comprising a population of zeolite nanoparticles dispersed therein disposed on the nonwoven substrate,

wherein the zeolite nanoparticles comprise an effective amount of antimicrobial metal ions to kill or inhibit the growth of a microbe.

62. The sheet of embodiment 61, wherein the zeolite nanoparticles are hydrophobically modified.
63. The sheet of embodiment 62, wherein the hydrophobically modified zeolite nanoparticles comprise a surface that has been modified via association of a hydrophobic capping molecule.
64. The sheet of embodiment 63, wherein the capping molecule comprises a hydrophobic molecule comprising a cationic moiety, and wherein the cationic moiety is electrostatically associated with the surface of the zeolite.
65. The sheet of embodiment 64, wherein the capping molecule comprises an amine defined by Formula I or Formula II below

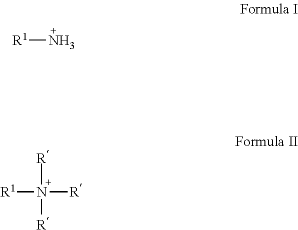

where

R1 is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

66. The sheet of embodiment 64, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.
67. The sheet of embodiment 64, wherein the surface is covalently modified via reaction with a halosilane selected from octadecyltrichlorosilane (OTS), octadecyltrichlorosilane (OTS), hexyltrichlorosilane (HTS), ethyltrichlorosilane (ETS), and combinations thereof.
68. The sheet of any of embodiments 61-67, wherein the zeolite nanoparticles have an average diameter of less than 100 nm, such as from 10 nm to less than 100 nm, or from 20 nm to 60 nm.
69. The sheet of any one of embodiments 61-68, wherein the antimicrobial metal ions comprise metal nanoparticles formed from an antimicrobial metal.
70. The sheet of embodiment 69, wherein the antimicrobial metal comprises silver, copper, zinc, or a combination thereof.
71. The sheet of any of embodiments 69-70, wherein the metal nanoparticles have an average diameter of 10 nm or less, such as from 1 nm to 10 nm, or from 1 nm to 5 nm.
72. The sheet of any of embodiments 69-71, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
73. The sheet of any one of embodiments 61-72, wherein the antimicrobial metal ions comprise antimicrobial metal ions retained at ion-exchangeable sites within the zeolite nanoparticles.
74. The sheet of embodiment 73, wherein the antimicrobial metal ions include copper ions, zinc ions, silver ions, or a combination thereof.
75. The sheet of any one of embodiments 73-74, wherein the antimicrobial metal ions are present in an amount of 10% or greater of the ion exchange capacity of the zeolite nanoparticles, such as from 50% up to 100% of the ion exchange capacity of the zeolite nanoparticles.
76. The sheet of any one of embodiments 61-75, wherein the zeolite nanoparticles have an average internal surface area of at least 300 m²/g.
77. The sheet of any one of embodiments 61-76, wherein the zeolite nanoparticles further comprise an adjuvant.
78. The sheet of embodiment 77, wherein the adjuvant includes a small molecule antimicrobial agent.
79. The sheet of any one of embodiments 61-78, wherein the zeolite nanoparticles are present in the transferrable carrier at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
80. The sheet of any one of embodiments 61-79, wherein the transferrable carrier further comprises a fabric softening material, a perfume, a fragrance, an antistatic compound, a soil release agent, an optical brightener, an odor control agent, a fiber lubricant, an antioxidant, a sunscreen, or any combination thereof.
81. The sheet of any one of embodiments 61-80, wherein the transferrable carrier comprises a wax.
82. The sheet of any one of embodiments 61-81, wherein when drying wet laundry in a tumble-type dryer in the presence of the sheet, at least a portion of the zeolite nanoparticles transfer from the nonwoven substrate to the laundry once the temperature within the tumble-type dryer is greater than about 120° F.
83. A method of applying an antimicrobial agent to a recipient textile, the method comprising drying the recipient textile in a tumble-type dryer in the present of the sheet of any of embodiments 61-82.
84. A textile comprising

a woven or nonwoven substrate; and

a population of zeolite nanoparticles disposed on the woven or nonwoven substrate,

wherein the zeolite nanoparticles comprise an effective amount of antimicrobial metal ions to kill or inhibit the growth of a microbe.

85. The textile of embodiment 84, wherein the woven or nonwoven substrate comprises a garment.
86. The textile of any of embodiments 84-85, wherein the woven or nonwoven substrate comprises a wound dressing.
87. The textile of any of embodiments 84-85, wherein the woven or nonwoven substrate comprises fibers formed from cotton, polyester, spandex, rayon, nylon, wool, silk, or a combination thereof.
88. The textile of any of embodiments 84-87, wherein the zeolite nanoparticles are covalently attached to the woven or nonwoven substrate.
89. The textile of any of embodiments 84-87, wherein the zeolite nanoparticles are non-covalently adsorbed to the woven or nonwoven substrate.
90. The textile of any of embodiments 84-89, wherein the zeolite nanoparticles are hydrophobically or hydrophilically modified.
91. The textile of any of embodiments 84-90, wherein the zeolite nanoparticles have an average diameter of less than 100 nm, such as from 10 nm to less than 100 nm, or from 20 nm to 60 nm.
92. The textile of any one of embodiments 84-91, wherein the antimicrobial metal ions comprise metal nanoparticles formed from an antimicrobial metal.
93. The textile of embodiment 92, wherein the antimicrobial metal comprises silver, copper, zinc, or a combination thereof.
94. The textile of any of embodiments 92-93, wherein the metal nanoparticles have an average diameter of 10 nm or less, such as from 1 nm to 10 nm, or from 1 nm to 5 nm.
95. The textile of any of embodiments 92-94, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
96. The textile of any one of embodiments 84-95, wherein the antimicrobial metal ions comprise antimicrobial metal ions retained at ion-exchangeable sites within the zeolite nanoparticles.
97. The textile of embodiment 96, wherein the antimicrobial metal ions include copper ions, zinc ions, silver ions, or a combination thereof.
98. The textile of any one of embodiments 96-97, wherein the antimicrobial metal ions are present in an amount of 10% or greater of the ion exchange capacity of the zeolite nanoparticles, such as from 50% up to 100% of the ion exchange capacity of the zeolite nanoparticles.
99. The textile of any one of embodiments 84-98, wherein the zeolite nanoparticles have an average internal surface area of at least 300 m²/g.
100. The textile of any one of embodiments 84-99, wherein the zeolite nanoparticles further comprise an adjuvant.
101. The textile of embodiment 100, wherein the adjuvant includes a small molecule antimicrobial agent.
102. The textile of any one of embodiments 84-100, wherein the zeolite nanoparticles are present on the substrate at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
103. A hemostatic composition comprising:

a binder; and

a population of zeolite nanoparticles dispersed therein.

104. The composition of embodiment 103, wherein the zeolite nanoparticles comprise an effective amount of antimicrobial metal ions to kill or inhibit the growth of a microbe.
105. The composition of any of embodiments 103-104, wherein the zeolite nanoparticles are hydrophobically or hydrophilically modified.
106. The composition of any of embodiments 103-105, wherein the zeolite nanoparticles have an average diameter of less than 100 nm, such as from 10 nm to less than 100 nm, or from 20 nm to 60 nm.
107. The composition of any one of embodiments 104-106, wherein the antimicrobial metal ions comprise metal nanoparticles formed from an antimicrobial metal.
108. The composition of embodiment 107, wherein the antimicrobial metal comprises silver, copper, zinc, or a combination thereof.
109. The composition of any of embodiments 107-108, wherein the metal nanoparticles have an average diameter of 10 nm or less, such as from 1 nm to 10 nm, or from 1 nm to 5 nm.
110. The composition of any of embodiments 107-109, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
111. The composition of any one of embodiments 104-110, wherein the antimicrobial metal ions comprise antimicrobial metal ions retained at ion-exchangeable sites within the zeolite nanoparticles.
112. The composition of embodiment 111, wherein the antimicrobial metal ions include copper ions, zinc ions, silver ions, or a combination thereof.
113. The composition of any one of embodiments 111-112, wherein the antimicrobial metal ions are present in an amount of 10% or greater of the ion exchange capacity of the zeolite nanoparticles, such as from 50% up to 100% of the ion exchange capacity of the zeolite nanoparticles.
114. The composition of any one of embodiments 103-113, wherein the zeolite nanoparticles 25 have an average internal surface area of at least 300 m²/g.
115. The composition of any one of embodiments 103-114, wherein the zeolite nanoparticles further comprise an adjuvant.
116. The composition of embodiment 115, wherein the adjuvant includes a small molecule antimicrobial agent.
117. The composition of any of embodiments 103-113, wherein the composition comprises a first population of zeolite nanoparticles and a second population of zeolite nanoparticles dispersed in the binder,

wherein the first population of zeolite nanoparticles comprises an effective amount of silver ions to kill or inhibit the growth of a microbe; and

wherein the second population of zeolite nanoparticles comprises an effective amount of calcium ion to enhance blood coagulation upon application of the composition to a wound.

118. The composition of embodiment 117, wherein the composition further comprises an additional population of zeolite nanoparticles dispersed in the binder,

wherein the additional population of zeolite nanoparticles comprises an effective amount of zinc ions to kill or inhibit the growth of a microbe.

118a. The composition of embodiment 117, wherein the composition further comprises an additional population of zeolite nanoparticles dispersed in the binder,

wherein the additional population of zeolite nanoparticles comprises an effective amount of copper ions to kill or inhibit the growth of a microbe.

119. The composition of any of embodiments 103-118, wherein the binder is clay-based, such as kaolin, kaolinite, bentonite, montmorillonite, or a combination thereof.
120. The composition of any of embodiments 103-119, the binder is of an irregularly-shaped granular form having a size distribution determined by sieving ground material with 40 mesh and 16 mesh cut-off screens.
121. A method for promoting blood coagulation comprising: applying a wound dressing, covering, or application system to a bleeding area, wherein the wound dressing, covering, or application system comprises:

a composition of any of embodiments 103-120; and

a gauze pad, a multiple layer cover, or a permeable bandage.

122. A method for promoting blood coagulation comprising:

applying a composition of any of embodiments 103-120 to a bleeding area; and

applying a gauze pad, a multiple layer cover, or a permeable bandage to a bleeding area.

123. A method of clotting blood flowing from a wound, said method comprising the steps of:

applying a composition of any of embodiments 103-120 to said wound, said composition being capable of producing a controllable blood clotting effect on said wound; and

maintaining the composition in contact with said wound for an amount of time sufficient to cause blood flowing from said wound to clot.

124. A method of promoting wound healing, said method comprising:

applying a composition of any of embodiments 103-120 to said wound; and maintaining the composition in contact with the wound for a sufficient amount of time to promote wound healing, wound closure, and/or tissue growth and regeneration 125. An antimicrobial composition comprising water and a population of zeolite nanoparticles dispersed therein,

wherein the zeolite nanoparticles comprise
(i) an effective amount of antimicrobial metal ions to kill or inhibit the growth of a microbe; and
(ii) an optical tracer associated with the zeolite nanoparticles.
   126. The composition of embodiment 125, wherein the zeolite nanoparticles comprise a surface that has been modified via association of a hydrophobic capping molecule.
   127. The composition of embodiment 126, wherein the capping molecule comprises a hydrophobic molecule comprising a cationic moiety, and wherein the cationic moiety is electrostatically associated with the surface of the zeolite.
   128. The composition of embodiment 127, wherein the capping molecule comprises an amine defined by Formula I or Formula II below

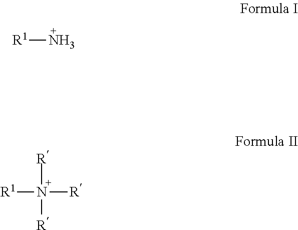

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

129. The composition of embodiment 126, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.
130. The composition of embodiment 126, wherein the surface is covalently modified via reaction with a halosilane selected from octadecyltrichlorosilane (OTS), hexyltrichlorosilane (HTS), ethyltrichlorosilane (ETS), and combinations thereof.
131. The composition of any of embodiments 125-130, wherein the zeolite nanoparticles have an average diameter of less than 100 nm, such as from 10 nm to less than 100 nm, or from 20 nm to 60 nm.
132. The composition of any one of embodiments 125-131, wherein the antimicrobial metal ions comprise metal nanoparticles formed from an antimicrobial metal.
133. The composition of embodiment 132, wherein the antimicrobial metal comprises silver, copper, zinc, or a combination thereof.
134. The composition of any of embodiments 132-133, wherein the metal nanoparticles have an average diameter of 10 nm or less, such as from 1 nm to 10 nm, or from 1 nm to 5 nm.
135. The composition of any of embodiments 132-134, wherein the metal nanoparticles are 25 present in an amount of at least 1% by weight, based on the total weight of the zeolite nanoparticles and the metal nanoparticles, such as from 1% to 25% by weight, based on the total weight of the zeolite nanoparticles and the metal nanoparticles.
136. The composition of any one of embodiments 125-135, wherein the antimicrobial metal ions comprise antimicrobial metal ions retained at ion-exchangeable sites within the zeolite nanoparticles.
137. The composition of embodiment 136, wherein the antimicrobial metal ions include copper ions, zinc ions, silver ions, or a combination thereof.
138. The composition of any one of embodiments 136-137, wherein the antimicrobial metal ions are present in an amount of 10% or greater of the ion exchange capacity of the zeolite nanoparticles, such as from 50% up to 100% of the ion exchange capacity of the zeolite nanoparticles.
139. The composition of any one of embodiments 125-138, wherein the zeolite nanoparticles have an average internal surface area of at least 300 m²/g.
140. The composition of any one of embodiments 125-139, wherein the zeolite nanoparticles further comprise an adjuvant.
141. The composition of embodiment 140, wherein the adjuvant includes a small molecule antimicrobial agent.
142. The composition of any one of embodiments 125-141, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
143. The composition of any one of embodiments 125-142, wherein the composition further comprises a non-ionic or zwitterionic surfactant.
144. The composition of embodiment 143, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.
145. The composition of any one of embodiments 125-144, wherein the composition further comprises a binder polymer dissolved or dispersed in the water.
146. The composition of claim 145, wherein the binder polymer comprises a water-soluble polymer.
147. The composition of embodiment 146, wherein the binder polymer comprises a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof, hemicellulose or derivatives thereof, alginate; tetramethylene ether glycol; polyvinyl pyrrolidone; polyvinyl esters such as polyvinyl acetate; copolymers thereof, and mixtures thereof.
148. The composition of any one of embodiments 146-147, wherein the binder polymer is present in an amount of 10% by weight or less, such as from 0.1% by weight to 10% by weight or from 0.1% to 5% by weight, based on the total weight of the composition.
149. The composition of any one of embodiments 125-148, wherein the optical tracer is covalently bound to the zeolite nanoparticles.
150. The composition of any one of embodiments 125-148, wherein the optical tracer is non-covalently associated with the zeolite nanoparticles.
151. The composition of any one of embodiments 125-150, wherein the optical tracer comprises a fluorophore.
152. The composition of embodiment 151, wherein the fluorophore comprises a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.
153. The composition of any one of embodiments 125-152, wherein the composition further comprises a co-solvent.
154. The composition of any one of embodiments 125-153, wherein the composition comprises an antimicrobial spray.
155. An antimicrobial composition comprising water, a water-soluble binder polymer, and a population of zeolite nanoparticles dispersed therein, wherein the zeolite nanoparticles comprise an effective amount of antimicrobial metal ions to kill or inhibit the growth of a microbe.
156. The composition of embodiment 155, wherein the zeolite nanoparticles comprise a surface that has been modified via association of a hydrophobic capping molecule.
157. The composition of embodiment 156, wherein the capping molecule comprises a hydrophobic molecule comprising a cationic moiety, and wherein the cationic moiety is electrostatically associated with the surface of the zeolite.
157. The composition of embodiment 157, wherein the capping molecule comprises an amine defined by Formula I or Formula II below

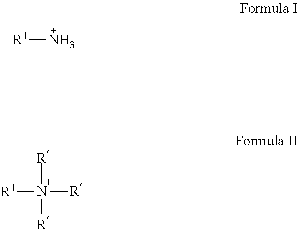

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

158. The composition of embodiment 156, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.
159. The composition of embodiment 158, wherein the surface is covalently modified via reaction with a halosilane selected from octadecyltrichlorosilane (OTS), hexyltrichlorosilane (HTS), ethyltrichlorosilane (ETS), and combinations thereof.
160. The composition of any of embodiments 155-159, wherein the zeolite nanoparticles have an average diameter of less than 100 nm, such as from 10 nm to less than 100 nm, or from 20 nm to 60 nm.
161. The composition of any one of embodiments 155-160, wherein the antimicrobial metal ions comprise metal nanoparticles formed from an antimicrobial metal.
162. The composition of embodiment 161, wherein the antimicrobial metal comprises silver, copper, zinc, or a combination thereof.
163. The composition of any of embodiments 161-162, wherein the metal nanoparticles have an average diameter of 10 nm or less, such as from 1 nm to 10 nm, or from 1 nm to 5 nm.
164. The composition of any of embodiments 161-163, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
165. The composition of any one of embodiments 155-164, wherein the antimicrobial metal ions comprise antimicrobial metal ions retained at ion-exchangeable sites within the zeolite nanoparticles.
166. The composition of embodiment 165, wherein the antimicrobial metal ions include copper ions, zinc ions, silver ions, or a combination thereof.
167. The composition of any one of embodiments 165-166, wherein the antimicrobial metal ions are present in an amount of 10% or greater of the ion exchange capacity of the zeolite nanoparticles, such as from 50% up to 100% of the ion exchange capacity of the zeolite nanoparticles.
168. The composition of any one of embodiments 155-167, wherein the zeolite nanoparticles have an average internal surface area of at least 300 m²/g.
169. The composition of any one of embodiments 155-168, wherein the zeolite nanoparticles further comprise an adjuvant.
170. The composition of embodiment 169, wherein the adjuvant includes a small molecule antimicrobial agent.
171. The composition of any one of embodiments 155-170, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
172. The composition of any one of embodiments 155-171, wherein the composition further comprises a non-ionic or zwitterionic surfactant.
173. The composition of embodiment 172, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.
174. The composition of any one of embodiments 155-173, wherein the binder polymer comprises a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof, hemicellulose or derivatives thereof, alginate; tetramethylene ether glycol; polyvinyl pyrrolidone; polyvinyl esters such as polyvinyl acetate; copolymers thereof, and mixtures thereof.
175. The composition of any one of embodiments 155-174, wherein the binder polymer is present in an amount of 10% by weight or less, such as from 0.1% by weight to 10% by weight or from 0.1% to 5% by weight, based on the total weight of the composition.
176. The composition of any one of embodiments 155-175, wherein the zeolite nanoparticles further comprise an optical tracer associated with the zeolite nanoparticles.
177. The composition of embodiment 176, wherein the optical tracer is covalently bound to the zeolite nanoparticles.
178. The composition of embodiment 176, wherein the optical tracer is non-covalently associated with the zeolite nanoparticles.
179. The composition of any one of embodiments 176-178, wherein the optical tracer comprises a fluorophore.
180. The composition of embodiment 179, wherein the fluorophore comprises a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.
181. The composition of any one of embodiments 155-180, wherein the composition further comprises a cosolvent.
182. The composition of any one of embodiments 155-181, wherein the composition comprises an antimicrobial spray.
183. A method of producing a coating on a surface comprising:

(a) applying to the surface the composition of any of embodiments 125-182; and

(b) allowing the composition to dry to produce the coating.

184. The method of embodiment 183, wherein the coating kills or inhibits the growth of a microbe.
185. The method of any one of embodiments 183-184, wherein the microbe is selected from a bacteria, a fungi, a virus, an algae, or a combination thereof.
186. The method of embodiment 185, wherein the microbe is a bacteria selected from Escherichia coli, Staphylococcus aureus, Bacillus coagulans, Bacillus megaterium, Bacillus subtilis, Enterococcus faecium, Pseudoxanthomonas spp., Pseudomonas putida, Pseudomonas aeruginosa, Pseudomonas maculicola, Pseudomanas chlororaphis, Pseudomonas flourescens, Nocardia brasiliensis, Nocardia globerula, Acinetobacter genomospecies, Acinetobacter calcoaceticus, Acinetobacter baumannii, Stenotrophomonas maltophlia, Pantoea stewarti ss stewarti, Chryseobacterium balustinus, Duganella zoogloeoides, Chryseobacterium meningosepticum, Staphylococcus hominis, Nocardia transvalensis, Burkolderia glumea, Pediococcus acidilactici/parvulus, Sphingomonas terrae, Corynebacterium spp., Gordonia rubripertincta, Rhodococcus rhodnii, Brevundimonas vesicularis, Providencian heimbachae, Gordonia sputi, Cellulosimicrobium cellulans, Sphingomonas sanguinis, Hydrogenophaga pseudoflava, Actinomadura cremea, Xanthomonas spp. or a combination thereof.
187. The method of embodiment 185, wherein the microbe is a fungi selected from Candida albicans, Candida parapsilosis, Candida tropicalis, Candida glabrata, Kluyveromyces marxianus, Hyphopichia burtani, Fusarium oxysporum, Botrytis cinerea, Aspergillus niger, Alternaria alternata, Sclerotinia sclerotiorum, Paecilomyces lilacinus, Penicillium vinaceum, Penicillium expansum, Penicillium charlesii, Penicillium expansum, or a combination thereof.
188. The method of embodiment 185, wherein the microbe is a coronavirus, such as SARS-CoV-2.
189. The method of any one of embodiments 183-188, wherein the composition comprises a binder polymer, and wherein the coating exhibits improved resistance to abrasion as compared to a coating formed from an otherwise identical composition in which the binder polymer is absent.
190. The method of any one of embodiments 183-189, wherein the composition comprises an optical tracer associated with the zeolite nanoparticles, and wherein the method further comprises interrogating the optical tracer to determine when the coating has been worn from the surface.
191. The method of embodiment 190, wherein interrogating the optical tracer comprises irradiating the surface with a UV light and visually observing fluorescence from the optical tracer to determine when the coating has been worn from the surface.
192. The method of any of embodiments 190-191, wherein the method further comprises replying the coating upon a determination the coating has been worn from the surface.
193. A method of forming a viricidal coating on a surface, the method comprising

(a) applying to the surface the composition comprising a population of zeolite nanoparticles dispersed in a carrier, wherein the zeolite nanoparticles comprise an effective amount of antimicrobial metal ions to kill or inhibit the growth of a virus; and

(b) allowing the composition to dry to produce the viricidal coating.

194. The method of embodiment 193, wherein the carrier comprises water.
195. The method of any one of embodiments 193-194, wherein the zeolite nanoparticles comprise a surface that has been modified via association of a hydrophobic capping molecule.
196. The method of embodiment 195, wherein the capping molecule comprises a hydrophobic molecule comprising a cationic moiety, and wherein the cationic moiety is electrostatically associated with the surface of the zeolite.
197. The method of embodiment 196, wherein the capping molecule comprises an amine defined by Formula I or Formula II below

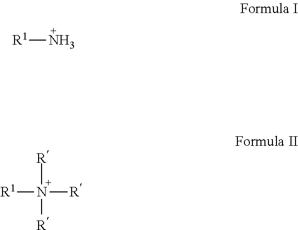

where

R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;

R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and

each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

198. The method of embodiment 195, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.
199. The method of embodiment 195, wherein the surface is covalently modified via reaction with a halosilane selected from octadecyltrichlorosilane (OTS), hexyltrichlorosilane (HTS), ethyltrichlorosilane (ETS), and combinations thereof.
200. The method of any of embodiments 193-199, wherein the zeolite nanoparticles have an average diameter of less than 100 nm, such as from 10 nm to less than 100 nm, or from 20 nm to 60 nm.
201. The method of any one of embodiments 193-200, wherein the antimicrobial metal ions comprise metal nanoparticles formed from an antimicrobial metal.
202. The method of embodiment 201, wherein the antimicrobial metal comprises silver, copper, zinc, iron, or a combination thereof.
203. The method of any of embodiments 201-202, wherein the metal nanoparticles have an average diameter of 10 nm or less, such as from 1 nm to 10 nm, or from 1 nm to 5 nm.
204. The method of any of embodiments 201-203, wherein the metal nanoparticles are present in an amount of at least 1% by weight, based on the total weight of the zeolite nanoparticles and the metal nanoparticles, such as from 1% to 25% by weight, based on the total weight of the zeolite nanoparticles and the metal nanoparticles.
205. The method of any one of embodiments 193-204, wherein the antimicrobial metal ions comprise antimicrobial metal ions retained at ion-exchangeable sites within the zeolite nanoparticles.
206. The method of embodiment 205, wherein the antimicrobial metal ions include copper ions, zinc ions, silver ions, iron ions, or a combination thereof.
207. The method of any one of embodiments 205-206, wherein the antimicrobial metal ions are present in an amount of 10% or greater of the ion exchange capacity of the zeolite nanoparticles, such as from 50% up to 100% of the ion exchange capacity of the zeolite nanoparticles.
208. The method of any one of embodiments 193-207, wherein the zeolite nanoparticles have an average internal surface area of at least 300 m²/g.
209. The method of any one of embodiments 193-208, wherein the zeolite nanoparticles further comprise an adjuvant.
210. The method of embodiment 209, wherein the adjuvant includes a small molecule antimicrobial agent.
211. The method of any one of embodiments 193-210, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.
212. The method of any one of embodiments 193-211, wherein the composition further comprises a non-ionic or zwitterionic surfactant.
213. The method of embodiment 212, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.
214. The method of any one of embodiments 193-213, wherein the binder polymer comprises a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof, hemicellulose or derivatives thereof, alginate; tetramethylene ether glycol; polyvinyl pyrrolidone; polyvinyl esters such as polyvinyl acetate; copolymers thereof, and mixtures thereof.
215. The method of any one of embodiments 193-214, wherein the binder polymer is present in an amount of 10% by weight or less, such as from 0.1% by weight to 10% by weight or from 0.1% to 5% by weight, based on the total weight of the composition.
216. The method of any one of embodiments 193-215, wherein the zeolite nanoparticles further comprise an optical tracer associated with the zeolite nanoparticles.
217. The method of embodiment 216, wherein the optical tracer is covalently bound to the zeolite nanoparticles.
218. The method of embodiment 216, wherein the optical tracer is non-covalently associated with the zeolite nanoparticles.
219. The method of any one of embodiments 216-218, wherein the optical tracer comprises a fluorophore.
220. The method of embodiment 219, wherein the fluorophore comprises a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.
221. The method of any one of embodiments 193-220, wherein the composition further comprises a cosolvent.
222. The method of any one of embodiments 193-221, wherein the composition comprises an antimicrobial spray.
223. The method of any one of embodiments 193-222, wherein the viricidal coating exhibits activity against coronaviruses, such as SARS-CoV-2.
224. The method of any one of embodiments 193-223, wherein the method further comprises contacting the surface with an oxidant, such as hydrogen peroxide.
225. The method of embodiment 224, wherein the hydrogen peroxide is applied as an aerosol.
226. The method of any one of embodiments 224-225, wherein transition metal ions present in the zeolite nanoparticle induce the formation of reactive radicals which kill or inhibit microbes

The compositions and methods of the appended claims are not limited in scope by the specific compositions described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compositions and methods disclosed herein are specifically described, other combinations of the components and steps described herein also are intended to fall within the scope of the appended claims, even if not specifically recited. Thus, a combination of components, steps, or constituents may be explicitly mentioned herein or less, however, other combinations of components, steps, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference. Unless otherwise specifically described, all percentages included herein are percentages by weight, based on total weight of the composition, excluding any propellant that may be present.

What is claimed is:

1. An antimicrobial composition comprising water and a population of zeolite nanoparticles dispersed therein,
wherein the zeolite nanoparticles comprise
(i) an effective amount of antimicrobial metal ions to kill or inhibit the growth of a microbe; and
(ii) an optical tracer associated with the zeolite nanoparticles.

2. The composition of claim 1, wherein the zeolite nanoparticles comprise a surface that has been modified via association of a hydrophobic capping molecule.

3. The composition of claim 2, wherein the capping molecule comprises a hydrophobic molecule comprising a cationic moiety, and wherein the cationic moiety is electrostatically associated with the surface of the zeolite.

4. The composition of claim 3, wherein the capping molecule comprises an amine defined by Formula I or Formula II below 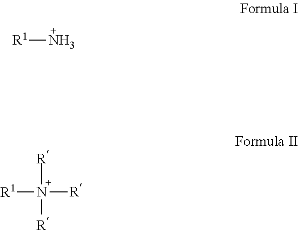
where
R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;
R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

5. The composition of claim 2, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

6. The composition of claim 2, wherein the surface is covalently modified via reaction with a halosilane selected from octadecyltrichlorosilane (OTS), hexyltrichlorosilane (HTS), ethyltrichlorosilane (ETS), and combinations thereof.

7. The composition of any of claims 1-6, wherein the zeolite nanoparticles have an average diameter of less than 100 nm, such as from 10 nm to less than 100 nm, or from 20 nm to 60 nm.

8. The composition of any one of claims 1-7, wherein the antimicrobial metal ions comprise antimicrobial metal ions retained at ion-exchangeable sites within the zeolite nanoparticles.

9. The composition of claim 8, wherein the antimicrobial metal ions include copper ions, zinc ions, silver ions, or a combination thereof.

10. The composition of any one of claims 8-9, wherein the antimicrobial metal ions are present in an amount of 10% or greater of the ion exchange capacity of the zeolite nanoparticles, such as from 50% up to 100% of the ion exchange capacity of the zeolite nanoparticles.

11. The composition of any one of claims 1-10, wherein the zeolite nanoparticles have an average internal surface area of at least 300 m²/g.

12. The composition of any one of claims 1-11, wherein the zeolite nanoparticles further comprise an adjuvant.

13. The composition of claim 12, wherein the adjuvant includes a small molecule antimicrobial agent.

14. The composition of any one of claims 1-13, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

15. The composition of any one of claims 1-14, wherein the composition further comprises a non-ionic or zwitterionic surfactant.

16. The composition of claim 15, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

17. The composition of any one of claims 1-16, wherein the composition further comprises a binder polymer dissolved or dispersed in the water.

18. The composition of claim 17, wherein the binder polymer comprises a water-soluble polymer.

19. The composition of claim 18, wherein the binder polymer comprises a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof; hemicellulose or derivatives thereof, alginate; tetramethylene ether glycol; polyvinyl pyrrolidone; polyvinyl esters such as polyvinyl acetate; copolymers thereof; and mixtures thereof.

20. The composition of any one of claims 17-19, wherein the binder polymer is present in an amount of 10% by weight or less, such as from 0.1% by weight to 10% by weight or from 0.1% to 5% by weight, based on the total weight of the composition.

21. The composition of any one of claims 1-20, wherein the optical tracer is covalently bound to the zeolite nanoparticles.

22. The composition of any one of claims 1-20, wherein the optical tracer is non-covalently associated with the zeolite nanoparticles.

23. The composition of any one of claims 1-22, wherein the optical tracer comprises a fluorophore.

24. The composition of claim 23, wherein the fluorophore comprises a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.

25. The composition of any one of claims 1-24, wherein the composition further comprises a co-solvent.

26. The composition of any one of claims 1-21, wherein the composition comprises an antimicrobial spray.

27. An antimicrobial composition comprising water, a binder polymer, and a population of zeolite nanoparticles dispersed therein,
wherein the zeolite nanoparticles comprise an effective amount of antimicrobial metal ions to kill or inhibit the growth of a microbe.

28. The composition of claim 27, wherein the zeolite nanoparticles comprise a surface that has been modified via association of a hydrophobic capping molecule.

29. The composition of claim 28, wherein the capping molecule comprises a hydrophobic molecule comprising a cationic moiety, and wherein the cationic moiety is electrostatically associated with the surface of the zeolite.

30. The composition of claim 28, wherein the capping molecule comprises an amine defined by Formula I or Formula II below 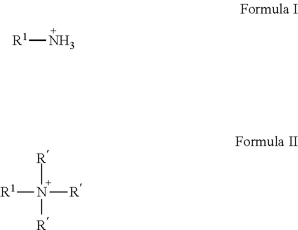
where
R¹ is selected from C₁₋₂₀ alkyl, C₂₋₂₀ alkenyl, C₂₋₂₀ alkynyl, C₁₋₂₀ haloalkyl, C₃₋₁₀ cycloalkyl, 6-10 membered aryl, 5-10 membered heteroaryl, 4-20 membered heterocycloalkyl, C₃₋₁₀ cycloalkyl-C₁₋₁₀ alkylene, 4-10 membered heterocycloalkyl-C₁₋₁₀ alkylene, 6-10 membered aryl-C₁₋₁₀ alkylene, and 5-10 membered heteroaryl-C₁₋₁₀ alkylene, each optionally substituted with 1, 2, 3, or 4 independently selected R^{X} groups;
R′ is, individually for each occurrence, C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene, each optionally substituted with 1, 2, 3, or 4 independently selected R^{X} groups; and
each R^{X}, when present, is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆alkylcarbamyl, di(C₁₋₆alkyl)carbamyl, carboxy, C₁₋₆alkylcarbonyl, C₁₋₆alkoxycarbonyl, C₁₋₆alkylcarbonylamino, C₁₋₆alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

31. The composition of claim 28, wherein the surface is covalently modified via reaction with an alkoxysilane selected from methyl triethoxysilane, methyl trimethoxysilane, methyl triphenoxysilane, propyl triphenoxysilane, methyl tricyclopentoxysilane, propyl tricyclohexoxy silane, methyl tricyclooctoxysilane, propyl diethoxy phenoxysilane, methyl tripropoxysilane, methyl tri-n-amyloxysilane, propyl triisopropoxysilane, ethyl triethoxysilane, diethyl diethoxysilane, isopropyl triethoxysilane, n-butyl triethoxysilane, n-amyl triethoxysilane, n-amyl trimethoxysilane, phenyl triethoxysilane, cyclopentyl triethoxysilane, cyclohexyl triethoxysilane, cyclooctyl triethoxysilane, dimethyl diethoxysilane, methyl ethyl diethoxysilane, tri(n-propyl)ethoxysilane, n-propyl trimethoxysilane, n-propyl triethoxysilane, di(n-propyl)diethoxysilane, trimethyl ethoxysilane, diphenyl diethoxysilane, diethyl diethoxysilane, n-octyl triethoxysilane, methyl tri(methoxyethoxy)silane, propyl tri(ethoxyethoxy)silane, IH, 1H,2H,2H-perfluorooctyltriethoxysilane, trimethoxy(octadecyl)silane, triethoxy(octyl)silane, trialkoxycaprylylsilanes (e.g., trimethoxycaprylylsilane), (3-aminopropyl)triethoxysilane (APTES), [3-(methylamino)propyl]-trimethoxysilane, (3-mercaptopropyl)trimethoxysilane, (3-isocyanatopropyl)trimethoxysilane, (3-chloropropyl)triethoxysilane, (3-cyanopropyl)triethoxysilane, (3-glycidyloxypropyl)triethoxysilane, 3-(trimethoxysilyl)propyl methacrylate, 3-(trimethoxysilyl)propyl acrylate, trimethoxy(2-phenylethyl)silane, and combinations thereof.

32. The composition of claim 28, wherein the surface is covalently modified via reaction with a halosilane selected from octadecyltrichlorosilane (OTS), hexyltrichlorosilane (HTS), ethyltrichlorosilane (ETS), and combinations thereof.

33. The composition of any of claims 27-32, wherein the zeolite nanoparticles have an average diameter of less than 100 nm, such as from 10 nm to less than 100 nm, or from 20 nm to 60 nm.

34. The composition of any one of claims 27-33, wherein the antimicrobial metal ions comprise antimicrobial metal ions retained at ion-exchangeable sites within the zeolite nanoparticles.

35. The composition of claim 34, wherein the antimicrobial metal ions include copper ions, zinc ions, silver ions, or a combination thereof.

36. The composition of any one of claims 34-35, wherein the antimicrobial metal ions are present in an amount of 10% or greater of the ion exchange capacity of the zeolite nanoparticles, such as from 50% up to 100% of the ion exchange capacity of the zeolite nanoparticles.

37. The composition of any one of claims 27-36, wherein the zeolite nanoparticles have an average internal surface area of at least 300 m²/g.

38. The composition of any one of claims 27-37, wherein the zeolite nanoparticles further comprise an adjuvant.

39. The composition of claim 38, wherein the adjuvant includes a small molecule antimicrobial agent.

40. The composition of any one of claims 27-39, wherein the zeolite nanoparticles are present in the composition at a concentration of from 1 ppm to 10,000 ppm, such as from 20 ppm to 10,000 ppm, from 10 ppm to 2,500 ppm, from 10 ppm to 2,000 ppm, from 10 ppm to 1,500 ppm, from 250 ppm to 1,500 ppm, from 500 ppm to 1,500 ppm, from 10 ppm to 250 ppm, from 20 ppm to 250 ppm, or from 20 ppm to 100 ppm.

41. The composition of any one of claims 27-40, wherein the composition further comprises a non-ionic or zwitterionic surfactant.

42. The composition of claim 41, wherein the non-ionic surfactant is present in the composition at a concentration of from 1 ppm to 2,000 ppm, such as from 1 ppm to 1,500 ppm, from 1 ppm to 1,000 ppm, from 5 ppm to 2,000 ppm, from 5 ppm to 1,500 ppm, or from 5 ppm to 1,000 ppm.

43. The composition of any one of claims 27-42, wherein the binder polymer comprises a polyalkylene oxide, such as polyethylene oxide; polyacrylic acid; polyvinyl alcohol; cellulose or derivatives thereof such as hydroxyethyl cellulose, hydroxypropyl methylcellulose, or hydroxymethyl cellulose; starch or derivatives thereof, hemicellulose or derivatives thereof, alginate; tetramethylene ether glycol; polyvinyl pyrrolidone; polyvinyl esters such as polyvinyl acetate; copolymers thereof, and mixtures thereof.

44. The composition of any one of claims 27-43, wherein the binder polymer is present in an amount of 10% by weight or less, such as from 0.1% by weight to 10% by weight or from 0.1% to 5% by weight, based on the total weight of the composition

45. The composition of any one of claims 27-44, wherein the zeolite nanoparticles further comprise an optical tracer associated with the zeolite nanoparticles.

46. The composition of claim 45, wherein the optical tracer is covalently bound to the zeolite nanoparticles.

47. The composition of claim 45, wherein the optical tracer is non-covalently associated with the zeolite nanoparticles.

48. The composition of any one of claims 45-47, wherein the optical tracer comprises a fluorophore.

49. The composition of claim 48, wherein the fluorophore comprises a xanthene, such as a fluorescein and/or a rhodamine, a cyanine, a naphthylamine, a napthalamide, a coumarin, an acridine, N-(p-(2-benzoxazolyl)phenyl)maleimide, a benzoxazoles, a benzoxadiazole, a stilbene, a pyrene, a pyrazoline, a quantum dot, or a combination thereof.

50. The composition of any one of claims 27-49, wherein the composition further comprises a cosolvent.

51. The composition of any one of claims 27-50, wherein the composition comprises an antimicrobial spray.

52. An antimicrobial composition comprising water and a population of modified zeolite nanoparticles dispersed therein, wherein the zeolite nanoparticles comprise an effective amount of antimicrobial metal ions to kill or inhibit the growth of a microbe.

53. An antimicrobial composition comprising a hydrophobic carrier and a population of zeolite nanoparticles dispersed therein, wherein the zeolite nanoparticles comprise an effective amount of antimicrobial metal ions to kill or inhibit the growth of a microbe.

54. A method of producing a coating on a surface comprising:
(a) applying to the surface the composition of any of claims 1-53; and
(b) allowing the composition to dry to produce the coating.

55. A method of forming a viricidal coating on a surface, the method comprising
(a) applying to the surface the composition comprising a population of zeolite nanoparticles dispersed in a carrier, wherein the zeolite nanoparticles comprise an effective amount of antimicrobial metal ions to kill or inhibit the growth of a virus; and
(b) allowing the composition to dry to produce the viricidal coating.

56. A dryer sheet comprising
a nonwoven substrate; and
a transferrable carrier comprising a population of zeolite nanoparticles dispersed therein disposed on the nonwoven substrate,
wherein the zeolite nanoparticles comprise an effective amount of antimicrobial metal ions to kill or inhibit the growth of a microbe.

57. A textile comprising
a woven or nonwoven substrate; and
a population of zeolite nanoparticles disposed on the woven or nonwoven substrate,
wherein the zeolite nanoparticles comprise an effective amount of antimicrobial metal ions to kill or inhibit the growth of a microbe.

58. A hemostatic composition comprising:
a binder; and
a population of zeolite nanoparticles dispersed therein.

59. A method for promoting blood coagulation comprising: applying a wound dressing, covering, or application system to a bleeding area, wherein the wound dressing, covering, or application system comprises:
a population of zeolite nanoparticles; and
a gauze pad, a multiple layer cover, or a permeable bandage.

60. A method for promoting blood coagulation comprising:
applying a population of zeolite nanoparticles to a bleeding area; and
applying a gauze pad, a multiple layer cover, or a permeable bandage to a bleeding area.

61. A method of clotting blood flowing from a wound, said method comprising the steps of:
applying a population of zeolite nanoparticles to said wound, said composition being capable of producing a controllable blood clotting effect on said wound; and
maintaining the composition in contact with said wound for an amount of time sufficient to cause blood flowing from said wound to clot.

62. A method of promoting wound healing, said method comprising:
applying a population of zeolite nanoparticles to said wound; and
maintaining the composition in contact with the wound for a sufficient amount of time to promote wound healing, wound closure, and/or tissue growth and regeneration.
